(19) United States
(12) Patent Application Publication
(10) Pub. No.: US 2025/0136970 A1
(43) Pub. Date: May 1, 2025

(54) CONTINUOUS PRECIPITATION FOR MRNA PURIFICATION

(71) Applicant: ModernaTX, inc., Cambridge, MA (US)

(72) Inventor: Mark Geng, Cambridge, MA (US)

(73) Assignee: ModernaTX, inc., Cambridge, MA (US)

(21) Appl. No.: 18/835,106

(22) PCT Filed: Feb. 3, 2023
(86) PCT No.: PCT/US2023/012261

§ 371 Date: Aug. 1, 2024

Related U.S. Application Data
(60) Provisional application No. 63/306,212, filed on Feb. 3, 2022.

(51) Int. Cl. C12N 15/10

(52) U.S. Cl. CPC C12N 15/1017

(57) ABSTRACT

Aspects of the disclosure relate to methods of purifying mRNA by precipitating the mRNA, washing the precipitate to remove impurities and salts, and resuspending the washed mRNA to produce an mRNA composition. The disclosure describes reagents and methods useful for precipitation, washing, and resuspension of mRNA, and compositions produced by the methods described herein.

### RELATED APPLICATION

This application claims the benefit under 35 U.S.C. § 119(e) of U.S. provisional application No. 63/306,212, filed Feb. 3, 2022, the contents of which are hereby incorporated by reference herein in their entirety.

### BACKGROUND

Messenger RNA (mRNA) is an emerging alternative to conventional small molecule and protein therapeutics due to the potency and programmability of mRNA. mRNA encoding a desired therapeutic protein can be administered to a subject for in vivo expression of the protein to therapeutic effect, such as vaccination or replacement of a protein encoded by a mutated gene. However, improved methods of purifying produced mRNA are needed.

### SUMMARY

Provided herein are methods of purifying nucleic acids (e.g., mRNAs) by precipitation, filtration, and resuspension. Nucleic acids such as mRNA are commonly purified by chromatography-based methods (e.g., oligo-dT chromatography and/or high-performance liquid chromatography), but such methods are limited by the dynamic binding capacity of stationary phases used in chromatography. Exceeding the binding capacity of the column causes excess mRNA to flow through the column, resulting in lost mRNA and reduced efficiency of the manufacturing process. To address these challenges, mRNA is purified by precipitating dissolved mRNA, to form a composition containing solid mRNA, but in which impurities remain dissolved. Impurities are then removed by separating the liquid phase of the composition from the precipitated mRNA, such as by aspiration or filtration. Then, one or more washing processes are conducted, in which a washing solution is added to dilute soluble impurities, such that subsequent separation of this washing solution from the precipitated mRNA reduces the total abundance of soluble impurities associated with the mRNA. This washing process may be repeated multiple times, to serially dilute and remove impurities until a desired purity is achieved or no detectable impurities remain. Additionally, washing solutions may contain reagents useful for removing particular impurities from an mRNA composition, such as those used to produce the mRNA, but whose presence is undesired in a final mRNA composition. For example, DNA templates and RNA polymerases are useful for producing mRNA by in vitro transcription, but not for translation of mRNA in vivo, and so must be removed before downstream applications. Accordingly, DNases and proteases may be added to digest DNA templates and proteins (e.g., RNA polymerases) into DNA and peptide fragments, respectively, which may then be separated from precipitated mRNA by filtration. After removal of impurities, mRNA may be resolubilized and resuspended to produce an mRNA composition that contains fewer impurities than the initial mRNA composition, or no impurities, and is thus useful in downstream applications (e.g., administration for in vivo translation of an encoded protein). These methods may be performed in a continuous manner, where an mRNA is continuously input into a process, manipulated by intermediate steps, and output, without interruptions. In contrast to batch-based methods, which require loading inputs in discrete batches, and must be interrupted to transfer materials between steps and remove outputs. Thus, continuous methods of precipitating, filtering, washing, and resuspending mRNA allow removal of impurities in a faster, more streamlined manner than batch-based methods.

Accordingly, described herein, in some aspects, is a continuous method of removing one or more impurities from a composition comprising messenger ribonucleic acid (mRNA), the continuous method comprising:
(a) precipitating mRNA from the composition to form a precipitated mRNA composition;
(b) removing one or more impurities from the precipitated mRNA composition; and then
(c) resuspending the precipitated mRNA. In some embodiments, the precipitating comprises adding a high-salt buffer and/or alcohol to the composition comprising the mRNA to form the precipitated mRNA composition. In some embodiments, the removing one or more impurities comprises contacting the precipitated mRNA composition with a filter membrane. In some embodiments, the removing one or more impurities comprises contacting the precipitated mRNA composition with a washing solution. In some embodiments, the removing one or more impurities comprises contacting the precipitated mRNA composition with a second filter membrane. In some embodiments, the removing one or more impurities comprises contacting the precipitated mRNA composition with a second washing solution.

In some aspects, a continuous method described herein comprises:
(i) adding a high-salt buffer to the mRNA composition to form a precipitated mRNA composition;
(ii) contacting the precipitated mRNA composition with a filter membrane to remove one or more impurities from the precipitated mRNA composition;
(iii)(a) contacting the precipitated mRNA composition with a washing solution;
(iii)(b) contacting the precipitated mRNA composition with a second filter membrane to remove one or more impurities from the precipitated mRNA composition; and
(iv) contacting the precipitated mRNA composition with a resuspension solution to resuspend the mRNA.

In some embodiments, the high-salt buffer comprises a salt selected from the group consisting of lithium chloride, lithium acetate, lithium sulfate, sodium chloride, sodium acetate, sodium sulfate, ammonium chloride, ammonium acetate, and ammonium sulfate. In some embodiments, the high-salt buffer comprises ammonium sulfate. In some embodiments, the high-salt buffer has a salt concentration of about 0.1 M to about 5.0 M. In some embodiments, the high-salt buffer has a conductivity of 5 mS/cm or more, optionally wherein the high-salt buffer has a conductivity of about 5 mS/cm to about 300 mS/cm. In some embodiments, the precipitation step further comprises adding an alcohol to the mRNA composition. In some embodiments, the alcohol is ethanol or isopropanol. In some embodiments, the filter membrane is a tangential flow filtration (TFF) membrane. In some embodiments, the filter membrane is a hollow fiber membrane. In some embodiments, the filter membrane has a molecular weight cutoff of about 20 kDa to about 150 kDa.

In some embodiments, the washing solution comprises a surfactant, a salt, a DNase, and/or an RNase III.

In some embodiments, the washing solution comprises a surfactant. In some embodiments, the washing solution comprises a detergent. In some embodiments, the detergent is a Triton X-100 detergent.

In some embodiments, the washing solution comprises a salt. In some embodiments, the salt comprises a monovalent or divalent cation. In some embodiments, the monovalent or divalent cation is selected from the group consisting of lithium, sodium, ammonium, calcium, and magnesium. In some embodiments, comprises an anion selected from the group consisting of chloride, sulfate, and acetate. In some embodiments, the washing solution comprises sodium chloride, calcium chloride, and/or ammonium sulfate. In some embodiments, the salt concentration in the washing solution is between about 50 mM to about 800 mM.

In some embodiments, the washing solution comprises a Tris buffer. In some embodiments, the washing solution comprises EDTA. In some embodiments, the resuspension solution comprises an RNase inhibitor.

In some embodiments, the washing solution comprises a DNase. In some embodiments, the DNase is a DNase I. In some embodiments, the washing solution comprises an RNase III. In some embodiments, the washing solution comprises a protease. In some embodiments, the protease is proteinase K.

In some embodiments, the second filter membrane is a tangential flow filtration (TFF) membrane. In some embodiments, the second filter membrane is a hollow fiber membrane. In some embodiments, the second filter membrane has a molecular weight cutoff of about 20 kDa to about 150 kDa.

In some embodiments, the method further comprises repeating the steps of (iii)(a) and (iii)(b). In some embodiments, the steps of (iii)(a) and (iii)(b) are each performed 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, times prior to step (iv). In some embodiments, one or more repeats of step (iii)(a) uses a different washing solution than the first iteration of step (iii)(a). In some embodiments, the method comprises, prior to resuspending the mRNA, contacting the precipitated mRNA composition with a salt, surfactant, protease, DNase, and RNase III.

In some embodiments, the resuspension solution has a salt concentration of 20 mM or less, 15 mM or less, 10 mM or less, or 5 mM or less. In some embodiments, the resuspension solution comprises Tris. In some embodiments, the resuspension solution comprises EDTA. In some embodiments, the resuspension solution comprises an RNase inhibitor. In some embodiments, the resuspension solution has a pH of about 5.0 to about 7.5. In some embodiments, the resuspension solution has a conductivity of 10 mS/cm or less, 8 mS/cm or less, 6 mS/cm or less, 5 mS/cm or less, 4 mS/cm or less, 3 mS/cm or less, 2.5 mS/cm or less, 2.0 mS/cm or less, 1.5 mS/cm or less, 1.0 mS/cm or less, or 0.5 mS/cm or less.

In some aspects, the disclosure relates to purified mRNA compositions produced by any of the methods described herein, comprising a resuspended mRNA, where the resuspended mRNA comprises an open reading frame encoding a vaccine antigen or therapeutic protein. In some embodiments, the purified mRNA composition has a salt concentration of 20 mM or less, 15 mM or less, 10 mM or less, or 5 mM or less. In some embodiments, 1% or fewer, 0.8% or fewer, 0.6% or fewer, 0.5% or fewer, 0.4% or fewer, 0.3% or fewer, 0.2% or fewer, 0.1% or fewer, 0.05% or fewer, 0.04% or fewer, 0.03% or fewer, 0.02% or fewer, or 0.01% or fewer of the mRNA molecules in the purified mRNA composition are double-stranded RNA (dsRNA) molecules. In some embodiments, the concentration of double-stranded RNA (dsRNA) in the isolated mRNA composition is 1% (w/w) or less, 0.9% or less, 0.8% or less, 0.7% or less, 0.6% or less, 0.5% or less, 0.4% or less, 0.3% or less, 0.2% or less, 0.1% or less, 0.05% or less, 0.04% or less, 0.03% or less, 0.02% or less, 0.01% or less, or 0.008% or less, 0.006% or less, 0.004% or less, 0.002% or less, or 0.001% or less. In some embodiments, the purified mRNA composition has a protein concentration of 1% (%w/w) or less, 0.8% or less, 0.6% or less, 0.4% or less, or 0.2% or less. In some embodiments, the purified mRNA composition has a DNA concentration of 1% (%w/w) or less, 0.8% or less, 0.6% or less, 0.4% or less, or 0.2% or less. In some embodiments, at least 80%, at least 85%, at least 90%, at least 95%, at least 95%, at least 96%, at least 97%, at least 98%, at least 99%, or up to 100% of the mRNAs of the purified mRNA composition comprise a poly(A) tail.

### BRIEF DESCRIPTION OF THE DRAWINGS

FIG. 1 shows a schematic outlining an exemplary process of continuous mRNA purification by precipitation and filtration. mRNA and a high-salt buffer are mixed to precipitate the mRNA, the precipitated mRNA is filtered to remove soluble impurities, one or more washing solutions are added and filtered to remove any remaining impurities, and a resuspension solution is mixed with the precipitated mRNA to resuspend the precipitated mRNA, thereby producing a purified mRNA composition.

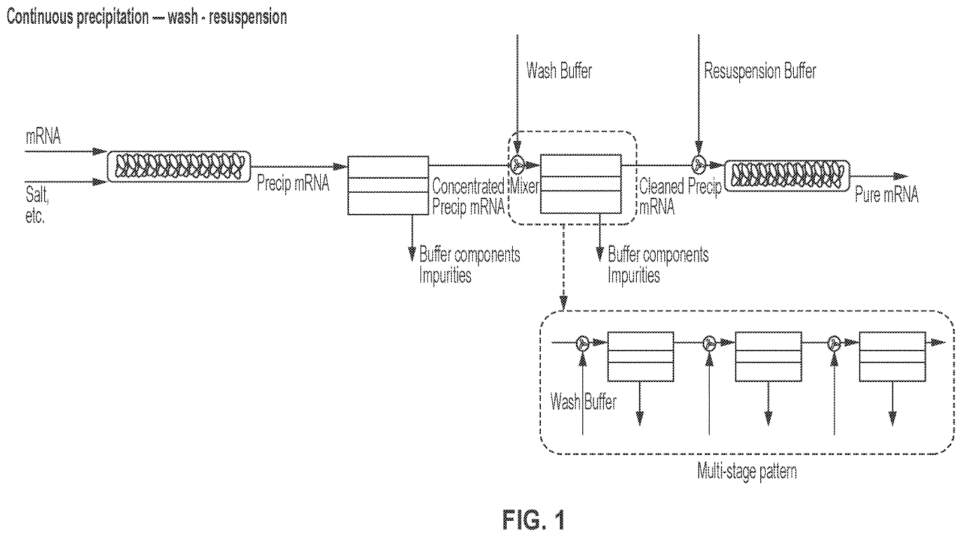

### DETAILED DESCRIPTION

Aspects of the disclosure relate to methods for purifying mRNA compositions by (i) precipitating the mRNA; (ii) removing the liquid phase of the composition, which contains soluble impurities, from the precipitated mRNA; (iii)(a) adding a washing solution to the precipitated mRNA; (iii)(b) removing the washing solution from the precipitated mRNA; and (iv) adding a resuspension solution to the precipitated mRNA to resuspend the mRNA. mRNA is useful for multiple applications, including expression of a desired protein in vivo, but mRNA production processes may introduce impurities that must be removed before the mRNA can be used in such applications. Purifying mRNAs by the methods described herein has multiple advantages, such as not being limited by the binding capacity of column stationary phases used in traditional chromatography approaches, and eliminating the need for chromatography reagents. Furthermore, multiple distinct washing solutions may be applied to a precipitated mRNA composition in sequence, to remove specific impurities from an mRNA composition, such as impurities known to be introduced by the manufacturing process (e.g., in vitro transcription enzymes and DNA templates), or impurities detected by analysis of an mRNA composition (e.g., dsRNAs). Finally, integration of precipitation, filtration, washing, and resuspension steps into a continuous method allows impurities to be removed from an mRNA composition in an automated manner, obviating the need for discrete input loading, intermediate transfer, and output release steps.

### Precipitation

Some aspects relate to methods of precipitating mRNAs in an mRNA composition to produce a precipitated mRNA composition comprising precipitated mRNA and soluble impurities. After the mRNA is precipitated, other components of the mRNA composition (e.g., proteins, DNA, free nucleotide triphosphates, short oligonucleotides) can be removed, e.g., by filtering the solution and washing the precipitated mRNA. After the precipitated mRNA is washed and filtered to remove impurities, the RNA can be resolubilized and dissolved in a solvent to produce a purified mRNA composition. The sugar-phosphate backbone of nucleic acids, such as mRNA, includes negatively charged phosphate ions, which makes individual mRNA molecules hydrophilic, allowing them to be dissolved in water. Adding a salt containing positive ions, such as sodium, ammonium, or lithium ions, neutralizes the negative charge of these phosphate groups, making mRNA molecules less hydrophilic and reducing the tendency of individual mRNA molecules to repel each other. Without being bound by theory, it is believed that this reduced solubility of mRNA, coupled with salt addition increasing the concentration of other solutes in the mRNA composition, can promote precipitation of mRNA, while other components of the solution remain dissolved.

In some embodiments, the methods described herein relate to precipitating mRNAs in an mRNA composition by adding a high-salt buffer to the composition. As described in the preceding paragraph, positive ions of a salt can balance the negative charge of phosphate moieties on the sugar: phosphate backbone of nucleic acids, which reduces the electrostatic repulsion between mRNA molecules and allows mRNA in a composition to be precipitated. A high-salt buffer for precipitating mRNAs in a composition may contain any salt, or multiple salts, that release positive ions when dissolved in water. Non-limiting examples of salts suitable for precipitating mRNAs include lithium chloride, sodium chloride, sodium acetate, ammonium acetate, calcium chloride, magnesium chloride, and ammonium sulfate. In some embodiments, the high-salt buffer comprises a salt that comprises a monovalent cation. A monovalent cation is a cation that comprises one more proton than electron, and thus has a 1+ charge. Non-limiting examples of monovalent cations include lithium ions, sodium ions, ammonium ions, and potassium ions. In some embodiments, the high-salt buffer solution comprises a salt that comprises a divalent cation. A divalent cation is a cation that comprises two more protons than electrons, and thus has a 2+ charge. Non-limiting examples of divalent cations include magnesium and calcium ions. In some embodiments, the high-salt buffer solution comprises a cation selected from the group consisting of a lithium ion, a sodium ion, a potassium ion, a ammonium ion, a magnesium ion, and a calcium ion; and an anion selected from the group consisting of sulfate, acetate, and chloride. In some embodiments, the high-salt buffer comprises a salt selected from the group consisting of lithium chloride, sodium chloride, sodium acetate, ammonium acetate, calcium chloride, magnesium chloride, and ammonium sulfate. In some embodiments, the high-salt buffer comprises lithium chloride. In some embodiments, the high-salt buffer comprises sodium chloride. In some embodiments, the high-salt buffer comprises sodium acetate. In some embodiments, the high-salt buffer comprises ammonium acetate. In some embodiments, the high-salt buffer comprises calcium chloride. In some embodiments, the high-salt buffer comprises magnesium chloride. In some embodiments, the high-salt buffer comprises ammonium sulfate.

High-salt buffers used to precipitate mRNAs may comprise any salt concentration that is sufficient to reduce the dielectric constant of an mRNA composition. In some embodiments, the high-salt buffer has a salt concentration of about 0.1 M to about 10 M. In some embodiments, the high-salt buffer has a salt concentration of 0.1 M to 0.2 M, 0.2 M to 0.4 M, 0.4 M to 0.6 M, 0.6M to 0.8 M, 0.8 M to 1.0 M, 1.0 M to 1.25 M, 1.25 M to 1.5 M, 1.5 M to 1.75 M, 1.75 M to 2.0M, 2.0 M to 2.25 M, 2.25 M to 2.5 M, 2.5 M to 2.75 M, 2.75 M to 3.0 M, 3.0 M to 3.5 M, 3.5 M to 4.0 M, 4.5 M to 5.0 M, 5.0 M to 6.0 M, 6.0 M to 6.5 M, 6.5 M to 7.0 M, 7.0 M to 7.5 M, 7.5M to 8.0 M, 8.5 M to 9.0 M, 9.0 M to 9.5 M, or 9.5 M to 10 M. In some embodiments, the high-salt buffer has a salt concentration of 0.1 M to 1.0 M. In some embodiments, the high-salt buffer has a salt concentration of 1.0 M to 2.0 M. In some embodiments, the high-salt buffer has a salt concentration of 2.0 M to 3.0 M. In some embodiments, the high-salt buffer has a salt concentration of 3.0 M to 4.0 M. In some embodiments, the high-salt buffer has a salt concentration of 4.0 M to 5.0 M. In some embodiments, the high-salt buffer has a salt concentration of about 1.0 M. In some embodiments, the high-salt buffer has a salt concentration of about 1.5 M. In some embodiments, the high-salt buffer has a salt concentration of about 2.0 M. In some embodiments, the high-salt buffer has a salt concentration of about 2.5 M. In some embodiments, the high-salt buffer has a salt concentration of about 3.0 M. In some embodiments, the high-salt buffer has a salt concentration of about 3.5 M. In some embodiments, the high-salt buffer has a salt concentration of about 4.0 M. In some embodiments, the high-salt buffer has a salt concentration of about 4.5 M. In some embodiments, the high-salt buffer has a salt concentration of about 5.0 M. In some embodiments, the salt concentration of a high-salt buffer refers to the concentration of a single salt. In other embodiments, the salt concentration of a high-salt buffer refers to the individual concentrations of two or more distinct salts. In other embodiments, the salt concentration of a high-salt buffer refers to the concentration of salt cations dissolved in the high-salt buffer.

In some embodiments, a high-salt buffer is added to an mRNA composition to increase the salt concentration of an mRNA composition to a certain level, or to a level exceeding a certain threshold. The salt concentration of a composition affects its dielectric constant, which in turn affect nucleic acid solubility, and so precipitation may, in some embodiments, be achieved by increasing the salt concentration of a composition to a particular level or above a certain threshold. In some embodiments, the salt concentration of the mRNA composition after adding the high-salt buffer is 0.1 M to 0.2 M, 0.2 M to 0.4 M, 0.4 M to 0.6 M, 0.6 M to 0.8 M, 0.8 M to 1.0 M, 1.0 M to 1.25 M, 1.25 M to 1.5 M, 1.5 M to 1.75 M, 1.75 M to 2.0 M, 2.0 M to 2.25 M, 2.25 M to 2.5 M, 2.5 M to 2.75 M, 2.75 M to 3.0 M, 3.0 M to 3.5 M, 3.5 M to 4.0 M, 4.5 M to 5.0 M, 5.0 M to 6.0 M, 6.0 M to 6.5 M, 6.5 M to 7.0 M, 7.0 M to 7.5 M, 7.5 M to 8.0 M, 8.5 M to 9.0 M, 9.0 M to 9.5 M, or 9.5 M to 10 M. In some embodiments, the salt concentration after high-salt buffer addition is 0.1 M to 1.0 M. In some embodiments, the salt concentration after high-salt buffer addition is 1.0 M to 2.0 M. In some embodiments, the salt concentration after high-salt buffer addition is 2.0 M to 3.0 M. In some embodiments, the salt concentration after high-salt buffer addition is 3.0 M to 4.0 M. In some embodiments, the salt concentration after high-salt buffer addition is 4.0 M to 5.0 M. In some embodiments, the salt concentration after high-salt buffer addition is about 1.0 M. In some embodiments, the salt concentration after high-salt buffer addition is about 1.5 M. In some embodiments, the salt concentration after high-salt buffer addition is about 2.0 M. In some embodiments, the salt concentration after high-salt buffer addition is about 2.5 M. In some embodiments, the salt concentration after high-salt buffer addition is about 3.0 M. In some embodiments, the salt concentration after high-salt buffer addition is about 3.5 M. In some embodiments, the salt concentration after high-salt buffer addition is about 4.0 M. In some embodiments, the salt concentration after high-salt buffer addition is about 4.5 M. In some embodiments, the salt concentration after high-salt buffer addition is about 5.0 M. The salt concentration following high-salt buffer addition may refer to the concentration of a single salt, total concentration obtained by adding the individual concentration of multiple salts present in the mRNA composition, or the concentration of salt cations dissolved in the mRNA composition.

In some embodiments, the high-salt buffer comprises a sugar. The presence of a sugar in a high-salt buffer for precipitating mRNA increases the total solute concentration of the buffer, which reduces the solubility of mRNA, thereby promoting nucleation, aggregation, and precipitation of the mRNA. Furthermore, the large size of sugar molecules allows them to act as volume exclusion agents, reducing the available space through which dissolved mRNAs can diffuse in solution, further promoting mRNA nucleation, aggregation, and precipitation. In some embodiments, the sugar is a monosaccharide. In some embodiments, the sugar is selected from the group consisting of glucose, fructose, mannose, and galactose. In some embodiments, the sugar is a disaccharide. In some embodiments, the disaccharide is trehalose, lactose, or sucrose.

In some embodiments, the sugar is a polysaccharide. In some embodiments, the polysaccharide is glycogen. In some embodiments, the high-salt buffer has a sugar concentration of about 0.1 M to about 10 M. In some embodiments, the high-salt buffer has a sugar concentration of 0.1 M to 0.2 M, 0.2 M to 0.4 M, 0.4 M to 0.6 M, 0.6 M to 0.8 M, 0.8 M to 1.0 M, 1.0 M to 1.25 M, 1.25 M to 1.5 M, 1.5 M to 1.75 M, 1.75 M to 2.0 M, 2.0 M to 2.25 M, 2.25 M to 2.5 M, 2.5 M to 2.75 M, 2.75 M to 3.0 M, 3.0 M to 3.5 M, 3.5 M to 4.0 M, 4.5 M to 5.0 M, 5.0 M to 6.0 M, 6.0 M to 6.5 M, 6.5 M to 7.0 M, 7.0 M to 7.5 M, 7.5 M to 8.0 M, 8.5 M to 9.0 M, 9.0 M to 9.5 M, or 9.5 M to 10 M. In some embodiments, the high-salt buffer has a sugar concentration of 0.1 M to 1.0 M.

In some embodiments, the high-salt buffer has a higher conductivity than the mRNA composition to which it is added, and therefore increases the conductivity of the mRNA composition following addition. In some embodiments, the high-salt buffer is added to increase the conductivity of a solution to a certain level, or above a certain threshold. The conductivity of a solution serves as a measure of its salt concentration, and which is negatively correlated with mRNA solubility. Thus, the conductivity of a high-salt buffer and/or mRNA composition following high-salt buffer addition measures the extent to which mRNA has been increased. In some embodiments, a high-salt buffer has a conductivity of at least 5 mS/cm, at least 6 mS/cm, at least 7 mS/cm, at least 8 mS/cm, at least 9 mS/cm, at least 10 mS/cm, at least 12 mS/cm, or at least 15 mS/cm. In some embodiments, a high-salt buffer has a conductivity of 5-10 mS/cm, 5-15 mS/cm, 5-7 mS/cm, 6-9 mS/cm, 8-10 mS/cm, 9-12 mS/cm, or 10-15 mS/cm. In some embodiments, a high-salt buffer has a conductivity of 5-300 mS/cm, 5-250 mS/cm, 5-200 mS/cm, 5-150 mS/cm, 5-100 mS/cm, 5-75 mS/cm, or 5-50 mS/cm. In some embodiments, after a high-salt buffer is added, the mRNA composition has a conductivity of at least 5 mS/cm, at least 6 mS/cm, at least 7 mS/cm, at least 8 mS/cm, at least 9 mS/cm, at least 10 mS/cm, at least 12 mS/cm, or at least 15 mS/cm. In some embodiments, after a high-salt buffer is added, the mRNA composition is added has a conductivity of 5-10 mS/cm, 5-15 mS/cm, 5-7 mS/cm, 6-9 mS/cm, 8-10 mS/cm, 9-12 mS/cm, or 10-15 mS/cm. In some embodiments, after a high-salt buffer is added, the mRNA composition is added has a conductivity of 5-300 mS/cm, 5-250 mS/cm, 5-200 mS/cm, 5-150 mS/cm, 5-100 mS/cm, 5-75 mS/cm, or 5-50 mS/cm.

In some embodiments, the precipitation step comprises adding an alcohol to the mRNA composition. Alcohols are less polar than water, and so the solubility of nucleic acids such as mRNA in alcohols is lower in alcohols than in water. Alcohols in alcohol-based solutions thus act as volume exclusion agents, reducing the available space through which mRNAs can diffuse, thereby promoting mRNA nucleation, aggregation, and precipitation. In some embodiments, the alcohol-containing solution comprises ethanol. In some embodiments, the alcohol-containing solution comprises isopropanol. In some embodiments, a solution added to mRNA during the precipitation step comprises at least 60% (%w/v), at least 65%, at least 70%, at least 75%, at least 80%, at least 85%, at least 90%, at least 95%, or up to 100% alcohol. In some embodiments, the alcohol-containing solution comprises about 70% alcohol. In some embodiments, the alcohol-containing solution comprises about 70% ethanol. In some embodiments, the alcohol-containing solution comprises about 70% isopropanol.

One benefit of the methods described herein is that precipitation may be used to convert most or all of the mRNA in a composition into a solid form before purification, and so precipitation-based methods are not limited by the binding capacity of a column. Precipitation may thus be carried out, in some embodiments, to precipitate a certain amount or percentage of mRNA in a composition. Methods of determining the percentage of mRNAs that are precipitated by a process are routine in the art. For example, the amount of dissolved mRNA present in a sample may be measured before and after precipitation, and the measured amounts may be compared to calculate the percentage of mRNAs that were precipitated. In some embodiments, at least 80% of the mRNAs in the mRNA composition are precipitated before liquid is removed from the composition. In some embodiments, at least 85% of the mRNAs in the mRNA composition are precipitated before liquid is removed from the composition. In some embodiments, at least 90% of the mRNAs in the mRNA composition are precipitated before liquid is removed from the composition. In some embodiments, at least 95% of the mRNAs in the mRNA composition are precipitated before liquid is removed from the composition. In some embodiments, at least 96% of the mRNAs in the mRNA composition are precipitated before liquid is removed from the composition. In some embodiments, at least 97% of the mRNAs in the mRNA composition are precipitated before liquid is removed from the composition. In some embodiments, at least 98% of the mRNAs in the mRNA composition are precipitated before liquid is removed from the composition. In some embodiments, at least 99% of the mRNAs in the mRNA composition are precipitated before liquid is removed from the composition. In some embodiments, up to 100% of the mRNAs in the mRNA composition are precipitated before liquid is removed from the composition.

### Filtration

In some embodiments, after the mRNA is precipitated, the composition containing the precipitated mRNA is filtered to separate the solution containing impurities.

Non-limiting examples of impurities that may be removed by filtration include salts (e.g., salts of an mRNA production process (e.g., in vitro transcription) and/or salts used to precipitate mRNA), proteins (e.g., in vitro transcription enzymes, DNases, proteinases, RNase III), and DNA from the precipitated mRNA. In some embodiments, the step of filtering comprises adding the precipitated mRNA and supernatant to a hollow fiber filter. A hollow fiber filter is a membrane comprising a network of fibers, and pores formed by the networked fibers. In contrast to tangential flow, in which a liquid or composition flows parallel to a TFF membrane, the supernatant and precipitated mRNA are applied to the hollow fiber filter such that the direction of liquid flow is through the filter (axial flow). Flow of the liquid may occur due to gravity, or be aided through the use of negative pressure below the filter, to facilitate filtration. In some embodiments, a vacuum is applied to facilitate the flow of liquid through the filter. Liquids, dissolved solutes, and suspended particles that are smaller than the pores of the filter pass through the pores, while solid components larger than the pores are retained above the filter. Thus, the precipitated mRNA is retained above the filter, while the supernatant passes through the filter. Following passage of the supernatant through the filter, a washing solution may be added to the precipitated mRNA to further remove any residual proteins or DNAs from the mRNA. In some embodiments, the hollow fiber filter has as a pore size of 10 μm or less, 9 μm or less, 8 μm or less, 7 μm or less, 6 μm or less, 5 μm or less, 4 μm or less, 3 μm or less, 2 μm or less, 1 μm or less, 0.9 μm or less, 0.8 μm or less, 0.7 μm or less, 0.6 μm or less, 0.5 μm or less, 0.4 μm or less, 0.3 μm or less, or 0.2 μm or less. In some embodiments, the hollow fiber filter has as a pore size of 30 μm or less. In some embodiments, the hollow fiber filter has as a pore size of 20 μm or less. In some embodiments, the hollow fiber filter has as a pore size of 10 μm or less. In some embodiments, the hollow fiber filter has as a pore size of 5 μm or less. In some embodiments, the hollow fiber filter has as a pore size of 2 μm or less. In some embodiments, the hollow fiber filter has as a pore size of 1 μm or less. In some embodiments, the hollow fiber filter has as a pore size of 0.5 μm or less.

In some embodiments, filtering mRNA is achieved by tangential flow filtration (TFF), which comprises contacting precipitated mRNA in an mRNA composition with a TFF membrane. In TFF, a mRNA composition flows over a filtration membrane (TFF membrane) comprising pores, with the pores of the membrane being oriented perpendicular to the direction of flow. Components of the mRNA composition flow through the pores, if able, while components that do not pass through the pores are retained in the mRNA composition. TFF thus removes smaller impurities, such as peptide fragments, DNA fragments, amino acids, and nucleotides from a mRNA composition, while larger molecules, such as full-length RNA transcripts, are retained in the mRNA composition. Additionally, RNA polymerases may produce double-stranded RNA transcripts during IVT, comprising an RNA: RNA hybrid of a full-length RNA transcript and another RNA with a complementary sequence. The second RNA that is hybridized to the full-length RNA transcript may be another full-length RNA, or a smaller RNA that hybridizes to only a portion of the full-length transcript. Like DNA fragments produced by DNase digestion of DNA templates, these small RNAs may also be removed during TFF, so that fewer dsRNA molecules are present in the filtered RNA composition.

The size of the pores of the TFF membrane affect which components are filtered (removed) from the mRNA composition and which are retained in the mRNA composition. Generally, TFF membranes are characterized in terms of a molecular weight cutoff, with components smaller than the molecular weight cutoff being removed from the mRNA composition during TFF, while components larger than the molecular weight cutoff being retained in the mRNA composition. In some embodiments, the tangential flow filtration comprises using a TFF membrane with a molecular weight cutoff of 500 kDa or less, 200 kDa or less, 150 kDa or less, 100 kDa or less, 50 kDa or less, 40 kDa or less, 30 kDa or less, 20 kDa or less, or lower. In some embodiments, the tangential flow filtration comprises using a TFF membrane with a molecular weight cutoff of 500 kDa or less. In some embodiments, the tangential flow filtration comprises using a TFF membrane with a molecular weight cutoff of 400 kDa or less. In some embodiments, the tangential flow filtration comprises using a TFF membrane with a molecular weight cutoff of 300 kDa or less. In some embodiments, the tangential flow filtration comprises using a TFF membrane with a molecular weight cutoff of 200 kDa or less. In some embodiments, the tangential flow filtration comprises using a TFF membrane with a molecular weight cutoff of 100 kDa or less. In some embodiments, the tangential flow filtration comprises using a TFF membrane with a molecular weight cutoff of 50 kDa or less. In some embodiments, the tangential flow filtration comprises using a TFF membrane with a molecular weight cutoff of 40 kDa or less. In some embodiments, the tangential flow filtration comprises using a TFF membrane with a molecular weight cutoff of 30 kDa or less. In some embodiments, the tangential flow filtration comprises using a TFF membrane with a molecular weight cutoff of 20 kDa or less. In some embodiments, the tangential flow filtration comprises using a TFF membrane with a molecular weight cutoff between 20 kDa and 200 kDa. In some embodiments, the tangential flow filtration comprises using a TFF membrane with a molecular weight cutoff between 20 kDa and 150 kDa.

Removal of liquid from an mRNA composition (e.g., by filtration) reduces the volume of the mRNA composition. When most or all of the mRNA in the composition is precipitated, not dissolved in the aqueous phase, little or no mRNA is removed by filtration. Thus, the loss of volume through filtration outweighs the loss of mRNA. Consequently, the concentration of mRNA is increased after filtration of a precipitated mRNA composition. Accordingly, in some embodiments, the concentration of mRNA in the precipitated mRNA composition is increased by filtration. In some embodiments, the concentration of mRNA in the precipitated mRNA composition is increased by about 2-10,000, 2-5,000, 2-2,500, 2-1,000, 2-500, 2-100, 10-10,000, 10-5,000, 10-2,500, 10-1,000, 10-500, 10-100, 100-100,000, 100-50,000, 100-25,000, 100-10,000, 100-5,000, 100-2,500, 100-1,500, 100-1,000, 100-500, or 100-200, relative to the concentration before filtration of the mRNA composition.

### Washing

Some aspects relate to methods of washing precipitated mRNA of an mRNA composition to remove one or more impurities from the precipitated mRNA. A washing solution refers to a solution in which the solubility of mRNA is minimal. Generally, a washing solution contains a salt that is capable of precipitating RNA, and/or an alcohol. After mRNA is precipitated, aspirating supernatant removes many dissolved impurities from the mRNA composition. However, the precipitated mRNA contains many bound salt cations, and any remaining liquid in the mRNA composition still contains residual impurities. Adding a washing solution dilutes these impurities, such that after the washing solution is removed, the total abundance of impurities of the mRNA composition is reduced. Where process of (a) adding a washing solution to precipitated mRNA to dilute impurities, and (b) removing the washing solution from the precipitated mRNA is repeated, the abundance of impurities in a precipitated mRNA composition is reduced during successive iterations, until the abundance of impurities is minimal. Removing impurities by repeated steps of washing precipitated mRNA and removing the washing solution has the additional advantage of being able to use different washing solutions in successive iterations, which may enhance the efficiency of impurity removal. For example, the first washing solution added to precipitated mRNA may be a washing solution in which the salts used to precipitate the mRNA are soluble, to reduce the salt concentration of the mRNA composition after precipitation. Next, precipitated mRNA may be washed with a second washing solution containing an enzyme (e.g., DNase, RNase III, or protease) to digest an impurity that may be present in the mRNA composition. This washing solution may contain one or more other components (e.g., an enzyme cofactor, such as magnesium ions) that promote enzyme activity, to enhance degradation of the impurity targeted by the enzyme. The next washing solution may then contain components useful for removing proteins (e.g., enzymes or host cell proteins) from a composition.

In some embodiments, the washing solution comprises an alcohol. Alcohols are generally less polar than water, and so the solubility of nucleic acids such as mRNA in alcohols is lower than in water. However, the relative reduction in salt solubility is less than the reduction in mRNA solubility, and so alcohol-based washing solutions may be used to dilute other impurities (e.g., salts, proteins, and/or DNA), allowing their removal when the liquid washing solution is separated from precipitated mRNA by aspiration or filtration. In some embodiments, the washing solution comprises ethanol. In some embodiments, the washing solution comprises isopropanol. In some embodiments, the washing solution comprises at least 60% (% w/v), at least 65%, at least 70%, at least 75%, at least 80%, at least 85%, at least 90%, at least 95%, or up to 100% alcohol. In some embodiments, the washing solution comprises about 70% alcohol. In some embodiments, the washing solution comprises about 70% ethanol. In some embodiments, the washing solution comprises about 70% isopropanol.

In some embodiments, the washing solution comprises a surfactant. A surfactant, as used herein, refers to a compound that reduces the surface tension between two liquids. Generally, surfactants promote the mixing between a first liquid and a second liquid that is less able, in the absence of the surfactant, to interact with the first liquid. For example, surfactants promote the interaction between inorganic solvents, such as water, and organic solvents, such as hydrocarbons or acetonitrile. When an organic solvent is present in an mRNA composition, such as when an organic solvent is used in an upstream process to separate mRNAs from other components of a mRNA composition, the use of surfactants is useful to promote interaction of organic solvents with the washing solution, and allow aspiration or filtration of the washing solution to remove organic solvents from precipitated mRNA. In some embodiments, the washing solution comprises a detergent. A detergent, as used herein, refers to a surfactant or combination of surfactants in an aqueous solution. Dilution of a surfactant in a detergent promotes interactions between surfactant molecules and water molecules, allowing water molecules of aqueous solutions (e.g., washing solutions) to bind indirectly to impurities in an mRNA composition, such that removal of the aqueous phase by filtration also removes surfactant-bound impurities. In some embodiments, the detergent is a Triton detergent, such as Triton X-100 detergent.

In some embodiments of the methods described herein, the washing solution comprises a salt. In addition to their uses in precipitating mRNA, the use of high-salt washing solutions is useful for removing proteins from a mRNA composition. Salt ions dissolved in water associate with oppositely charged moieties of proteins, and these associated salt ions attract water molecules, forming a bridge between water molecules and amino acids at the protein surface. By promoting indirect binding of water molecules to amino acids, salt ions thus increase the solubility of proteins in water. Removal of washing solutions in which proteins are highly soluble can therefore separate protein impurities from precipitated mRNA of an mRNA composition. Furthermore, salts present in a washing solution reduce the solubility of mRNA, as discussed above. The use of washing solutions containing one or more salts thus inhibits dissolution of precipitated mRNA during washing and filtration of washing solution, reducing the amount of mRNA lost during washing steps.

A washing solution for washing precipitated mRNAs and/or removing proteins from a composition may contain any salt, or multiple salts, that release positive and negative ions when dissolved in water. Washing solutions may contain the same salt used to precipitate the mRNA, or a different salt. Non-limiting examples of salts suitable for washing mRNAs include lithium chloride, sodium chloride, sodium acetate, ammonium acetate, calcium chloride, magnesium chloride, and ammonium sulfate. In some embodiments, the washing solution comprises a salt that comprises a monovalent cation. In some embodiments, the washing solution comprises a salt that comprises a divalent cation. In some embodiments, the washing solution comprises a cation selected from the group consisting of a lithium ion, a sodium ion, a potassium ion, a ammonium ion, a magnesium ion, and a calcium ion; and an anion selected from the group consisting of sulfate, acetate, and chloride. In some embodiments, the washing solution comprises a salt selected from the group consisting of lithium chloride, sodium chloride, sodium acetate, ammonium acetate, calcium chloride, magnesium chloride, and ammonium sulfate. In some embodiments, the washing solution comprises lithium chloride. In some embodiments, the washing solution comprises sodium chloride. In some embodiments, the washing solution comprises sodium acetate. In some embodiments, the washing solution comprises ammonium acetate. In some embodiments, the washing solution comprises calcium chloride. In some embodiments, the washing solution comprises magnesium chloride. In some embodiments, the washing solution comprises ammonium sulfate.

Washing solutions used to wash precipitated mRNAs and/or remove proteins from an mRNA composition may comprise any salt concentration that is sufficient to promote the solubility of proteins in water and/or inhibit dissolution of the precipitated mRNA. In some embodiments, the washing solution has a salt concentration of about 0.1 M to about 1.0 M. In some embodiments, the washing solution has a salt concentration of about 50 mM to about 800 mM. In some embodiments, the washing solution has a salt concentration of about 100 mM to about 600 mM. In some embodiments, the washing solution has a salt concentration of about 200 mM to about 500 mM. In some embodiments, the washing solution has a salt concentration of 0.1 M to 0.2 M, 0.2 M to 0.3 M, 0.3 M to 0.4 M, 0.4 M to 0.5 M, 0.5 M to 0.6 M, 0.6 M to 0.7

M, 0.7 M to 0.8 M, 0.8 M to 0.9 M, 0.9 M to 1.0 M, 1.0 M to 1.25 M, 1.25 M to 1.5 M, 1.5 M to 1.75 M, 1.75 M to 2.0 M, 2.0 M to 2.25 M, 2.25 M to 2.5 M, 2.5 M to 2.75 M, 2.75 M to 3.0 M, 3.0 M to 3.5 M, 3.5 M to 4.0 M, 4.5 M to 5.0 M, 5.0 M to 6.0 M, 6.0 M to 6.5 M, 6.5 M to 7.0 M, 7.0 M to 7.5 M, 7.5 M to 8.0 M, 8.5 M to 9.0 M, 9.0 M to 9.5 M, or 9.5 M to 10 M. In some embodiments, the washing solution has a salt concentration of 100 mM to 200 mM. In some embodiments, the washing solution has a salt concentration of 200 mM to 300 mM. In some embodiments, the washing solution has a salt concentration of 300 mM to 400 mM. In some embodiments, the washing solution has a salt concentration of 400 mM to 500 mM. In some embodiments, the washing solution has a salt concentration of 500 mM to 600 mM. In some embodiments, the washing solution has a salt concentration of 600 mM to 700 mM. In some embodiments, the washing solution has a salt concentration of 700 mM to 800 mM. In some embodiments, the washing solution has a salt concentration of 800 mM to 900 mM. In some embodiments, the washing solution has a salt concentration of 900 mM to 1.0 M. In some embodiments, the washing solution has a salt concentration of about 100 mM. In some embodiments, the washing solution has a salt concentration of about 150 mM. In some embodiments, the washing solution has a salt concentration of about 200 mM.In some embodiments, the washing solution has a salt concentration of about 250 mM. In some embodiments, the washing solution has a salt concentration of about 300 mM. In some embodiments, the washing solution has a salt concentration of about 400 mM. In some embodiments, the washing solution has a salt concentration of about 500 mM. In some embodiments, the salt concentration of a washing solution refers to the concentration of a single salt. In other embodiments, the salt concentration of a washing solution refers to the individual concentrations of two or more distinct salts. In other embodiments, the salt concentration of a washing solution refers to the concentration of salt cations dissolved in the high-salt buffer.

In some embodiments, the washing solution comprises a protease. Proteases are enzymes that catalyze the breakdown of proteins into smaller protein fragments, such as smaller polypeptides, oligopeptides, or individual amino acids. Generally, proteases catalyze hydrolysis of the peptide bonds that connect amino acids in polypeptide chains, with hydrolysis of a peptide bond resulting in the release of two polypeptides, two amino acids, or an amino acid and a polypeptide, depending on the site of cleavage. Exopeptidases are proteases that catalyze the peptide bond between a terminal amino acid in a polypeptide and an adjacent amino acid, resulting in the release of the terminal amino acid from the rest of the polypeptide. Endopeptidases are proteases that catalyze the peptide bond between internal amino acids in a polypeptide, releasing two separate polypeptides or oligopeptides. Endopeptidases may vary in specificity, cleaving more readily before or after certain amino acid residues. For example, trypsin readily cleaves after arginine or lysine, unless the arginine or lysine is followed by a proline. Cleaving “after” a first amino acid refers to cleavage of the peptide bond that connects the first amino acid to the next amino acid in a protein, with the protein being described by an amino acid sequence listing amino acids from the N-terminus to the C-terminus. Peptide fragments produced by protease-mediated cleavage of longer proteins are smaller than full-length mRNAs and more easily removed by filtration.

In some embodiments, the protease introduced into the mRNA composition is selected from the group consisting of proteinase K, Lys-C, trypsin, TPCK-treated trypsin, chymotrypsin, α-lytic protease, and endoproteinase AspN. In some embodiments, the protease is a serine protease, such as proteinase K. Proteinase K is an endopeptidase with broad specificity that cleaves the peptide bond adjacent to the carboxyl group of aliphatic and aromatic amino acids. Exemplary amino acids that can be serve as substrates for cleavage by proteinase K include alanine, glycine, isoleucine, leucine, proline, valine, tryptophan, tyrosine, and phenylalanine. An example of a DNA sequence encoding proteinase K is given by Accession No. X14688. An example of an amino acid sequence of proteinase K is given by Accession No. P06873. In some embodiments, the protease is proteinase K. In some embodiments, the protease is Lys-C. In some embodiments, the protease is TPCK-treated trypsin. In some embodiments, the protease is chymotrypsin. In some embodiments, the protease is an α-lytic protease. In some embodiments, the protease is AspN.

In some embodiments, after the protease is introduced into the mRNA composition, the concentration of the protease in the mRNA composition is about 0.1 Units/mL to about 100 Units/mL. With respect to a protein, a “Unit” (“U”) refers to an amount of the protein that is capable of performing a specific function in a given amount of time. For example, one unit of proteinase K is defined as the amount of enzyme required to liberate folin-positive amino acids and peptides corresponding to 1 μmol of tyrosine in 1 minute at 37° C. in a total reaction volume of 250 μL. See, e.g., Anson. J Gen Physiol. 1938. 22 (1): 79-89. In some embodiments, the concentration of protease in the mRNA composition is about 0.2 to about 50 Units/mL, about 0.3 to about 25 Units/mL, about 0.4 to about 10 Units/mL, about 0.5 to about 5 Units/mL, about 0.5 to about 3 Units/mL, about 0.5 to about 2 Units/mL, or about 0.5 to about 1 Unit/mL. In some embodiments, the concentration of protease in the mRNA composition is about 0.1 to about 2 Units/mL. In some embodiments, the concentration of protease in the mRNA composition is about 1 to about 10 Units/mL. In some embodiments, the concentration of protease in the mRNA composition is about 10 to about 100 Units/mL.

In some embodiments, the amount of the protease in the mRNA composition during the protease digestion step, relative to the amount of RNA polymerase in the mRNA composition, is at least 1:1,000,000 (1 Unit protease: 1,000,000 umol RNA polymerase). In some embodiments, the protease: RNA polymerase concentration in the mRNA composition is about 1:10 to about 1:100, about 1:100 to about 1:1,000, about 1:1,000 to about 1:10,000, about 1:10,000 to about 1:100,000, or about 1:100,000 to about 1:1,000,000. In some embodiments, the protease: RNA polymerase concentration in the mRNA composition is about 1:1,000 to about 1:50,000. In some embodiments, the amount of the protease in the mRNA composition during the protease digestion step, relative to the amount of other proteins in the mRNA composition, is at least 1:1,000,000 (1 Unit protease: 1,000,000 μmol other proteins). In some embodiments, the protease: protein concentration in the mRNA composition is about 1:10 to about 1:100, about 1:100 to about 1:1,000, about 1:1,000 to about 1:10,000, about 1:10,000 to about 1:100,000, or about 1:100,000 to about 1:1,000,000. In some embodiments, the protease: protein concentration in the mRNA composition is about 1:1,000 to about 1:50,000.

In some embodiments, the mRNA composition is incubated after protease addition, to allow sufficient time for protein digestion. In some embodiments, the step of protease digestion is conducted at about 37° C. In some embodiments, the protease digestion step is conducted at a temperature of 70° C. or lower, 60° C. or lower, 50° C. or lower, or 40° C. or lower. In some embodiments, the step of protease digestion is conducted for about 10 minutes to about 6 hours. In some embodiments, the step of protease digestion is conducted for at least 10 minutes, at least 15 minutes, at least 20 minutes, at least 30 minutes, at least 40 minutes, at least 50 minutes, or at least 60 minutes. In some embodiments, the step of protease digestion is conducted for at least 1 hour, at least 2 hours, at least 3 hours, at least 4 hours, or at least 5 hours. In some embodiments, the step of protease digestion is conducted for about 15 minutes. In some embodiments, the step of protease digestion is conducted for about 30 minutes. In some embodiments, the step of protease digestion is conducted for about 45 minutes. In some embodiments, the step of protease digestion is conducted for about 60 minutes.

In some embodiments, the IVT mRNA composition comprises one or more cations. In some embodiments, one or more cations are added to the IVT mRNA composition during or after IVT. The presence and concentration of cations can affect the activity of proteases. For example, some proteases are more active in the presence of divalent cations, such as magnesium (Mg2+) ions. Adding magnesium or other cations to a mRNA composition can thus improve the efficiency of protease digestion, thereby allowing for removal of more residual proteins from an IVT mRNA composition to produce a more pure RNA composition. In some embodiments, the cation present in or added to the mRNA composition is a magnesium ion. In some embodiments, the cation present in or added to the mRNA composition is a calcium ion. In some embodiments, the concentration of cations in the mRNA composition during protease digestion is about 10 mM to about 100 mM. In some embodiments, the concentration of cations is about 1 mM to about 5 mM, about 5 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, or about 90 to about 100 mM. In some embodiments, the concentration of cations is about 10 mM. In some embodiments, the concentration of cations is about 20 mM. In some embodiments, the concentration of cations is about 30 mM. In some embodiments, the concentration of magnesium ions in the mRNA composition during protease digestion is about 10 mM to about 100 mM. In some embodiments, the concentration of magnesium ions is about 1 mM to about 5 mM, about 5 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, or about 90 to about 100 mM. In some embodiments, the concentration of magnesium ions is about 10 mM. In some embodiments, the concentration of magnesium ions is about 20 mM. In some embodiments, the concentration of magnesium ions is about 30 mM.

In some embodiments, the washing solution comprises a DNase. DNases are enzymes that catalyze the cleavage of phosphodiester bonds between nucleotides in a DNA molecule, resulting in digestion of a DNA into multiple smaller DNA fragments. Addition of DNase thus allows digestion of these DNA templates into DNA fragments that are more easily removed by DNase digestion. After DNase digestion, the RNA transcripts are larger than the other components of the mRNA composition, and can thus be separated using size-based filtration methods, such as TFF and hollow fiber-based filtration methods described herein. In some embodiments, the DNase is DNase I.

In some embodiments, the DNase is incubated for a period of time sufficient to cleave one or more DNAs of the mRNA composition. A “period of time sufficient” to achieve an outcome refers to a length of time which, if allowed to pass, causes the outcome to be achieved. The period of time sufficient to cleave one or more DNAs, or to cleave a certain percentage of DNAs in a mRNA composition, may be determined by incubating DNase in a composition comprising DNA, sampling the composition after the passage of multiple periods of time, and determining the extent of DNA cleavage at each sampling time. If a given outcome has been achieved after the passage of a given period of time, that period of time is said to be sufficient to achieve the outcome. A period of time sufficient to cleave a certain percentage of DNAs refers to the period of time, after which at least that percentage of DNAs that were initially present at the start of the incubation have been cleaved by the DNase. Thus, after a period of time sufficient to cleave 80% of DNAs in a mRNA composition, at least 80% of DNAs that were initially present will have been cleaved by DNase. In some embodiments, the step of DNase digestion is conducted at about 37° C. In some embodiments, the DNase digestion step is conducted at a temperature of 70° C. or lower, 60° C. or lower, 50° C. or lower, or 40° C. or lower. In some embodiments, the step of DNase digestion is conducted for about 10 minutes to about 6 hours. In some embodiments, the step of DNase digestion is conducted for at least 10 minutes, at least 15 minutes, at least 20 minutes, at least 30 minutes, at least 40 minutes, at least 50 minutes, or at least 60 minutes. In some embodiments, the step of DNase digestion is conducted for at least 1 hour, at least 2 hours, at least 3 hours, at least 4 hours, or at least 5 hours. In some embodiments, the step of DNase digestion is conducted for about 15 minutes. In some embodiments, the step of DNase digestion is conducted for about 30 minutes. In some embodiments, the step of DNase digestion is conducted for about 45 minutes. In some embodiments, the step of DNase digestion is conducted for about 60 minutes.

In some embodiments, the washing solution comprises an RNase III. Ribonuclease III (RNase III) is an endoribonuclease that binds to and cleaves double-stranded RNA (dsRNA), and so it is useful for digesting dsRNAs produced during IVT to reduce the deleterious effects of dsRNA in downstream applications. Double-stranded RNA transcripts, in which at least a portion of an RNA transcript is hybridized to another RNA molecule, elicit an innate immune response when introduced into a cell, causing degradation of both strands of a dsRNA. Reducing the abundance of dsRNA molecules enables the production of less immunogenic, and thus more stable, RNA compositions. RNase III enzymes catalyze the cleavage of phosphodiester bonds between nucleotides in a dsRNA. In cells, RNase III enzymes typically produce shorter dsRNA fragments 18-25 base pairs in length, with each RNA strand having two nucleotides at the 3′ terminus that are not bound by complementary nucleotides on the opposing RNA strand. Such shorter dsRNA fragments play a role in RNA-mediated silencing of gene expression, and are known as siRNAs. These smaller dsRNA fragments produced by RNase III digestion are smaller than full-length single-stranded mRNA transcripts encoding proteins, and are thus more easily separated from desired mRNAs.

RNase III is expressed in many organisms and is highly conserved (e.g., Mian et al., Nucleic Acids Res., 1997, 25, 3187-95). RNase III species cloned to date contain an RNase III signature sequence and vary in size from 25 to 50 kDa. Multiple functions have been ascribed to RNase III. In both Escherichia coli and Saccharomyces cerevisiae, RNase III is involved in the processing of pre-ribosomal RNA (pre-rRNA) (e.g., Elela et al., Cell, 1996, 85, 115-24). RNase III is also involved in the processing of small molecular weight nuclear RNAs (snRNAs) and small molecular weight nucleolar RNAs (snoRNAs) in S. cerevisiae (e.g., Chanfreau et al., Genes Dev. 1996, 11, 2741-51; Qu et al., Mol. Cell. Biol. 1996, 19, 1144-58). In E. coli, RNase III is involved in the degradation of some mRNA species (e.g., Court et al., Control of messenger RNA stability, 1993, Academic Press, Inc, pp. 71-116). In some embodiments, the RNase III is an Escherichia coli, Thermotoga maritima, or Aquifex aeolicus RNase III.

In some embodiments, the RNase III is incubated for a period of time sufficient to cleave one or more dsRNAs in the mRNA composition. In some embodiments, the step of RNase III digestion is conducted at about 37° C. In some embodiments, the RNase III digestion step is conducted at a temperature of 70° C. or lower, 60° C. or lower, 50° C. or lower, or 40° C. or lower. In some embodiments, the step of RNase III digestion is conducted for about 10 minutes to about 6 hours. In some embodiments, the step of RNase III digestion is conducted for at least 10 minutes, at least 15 minutes, at least 20 minutes, at least 30 minutes, at least 40 minutes, at least 50 minutes, or at least 60 minutes. In some embodiments, the step of RNase III digestion is conducted for at least 1 hour, at least 2 hours, at least 3 hours, at least 4 hours, or at least 5 hours. In some embodiments, the step of RNase III digestion is conducted for about 15 minutes. In some embodiments, the step of RNase III digestion is conducted for about 30 minutes. In some embodiments, the step of RNase III digestion is conducted for about 45 minutes. In some embodiments, the step of RNase III digestion is conducted for about 60 minutes.

In some embodiments, the washing solution comprising an RNase III also comprises one or more ions that promote the activity of the RNase III. The presence and concentrations of different cations can affect the activity of an RNase III. For example, some RNase III enzymes are more active in the presence of divalent cations, such as magnesium (Mg2+). The cations present during RNase III digestion also affect the specificity of the enzyme. For example, certain RNAse III enzymes are more specific in the presence of magnesium ions compared to an equivalent concentration of other cations, such as manganese (Mn2+) ions. In some embodiments, the cation present in or added to the mRNA composition or composition is a magnesium ion. In some embodiments, the concentration of magnesium ions in the mRNA composition or composition during RNase III digestion is about 10 mM to about 100 mM. In some embodiments, the concentration of magnesium ions is about 1 mM to about 5 mM, about 5 mM to about 10 mM, about 10 mM to about 20 mM, about 20 mM to about 30 mM, about 30 mM to about 40 mM, about 40 mM to about 50 mM, about 50 mM to about 60 mM, about 60 mM to about 70 mM, about 70 mM to about 80 mM, about 80 mM to about 90 mM, or about 90 to about 100 mM. In some embodiments, the concentration of magnesium ions is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM. In some embodiments, the concentration of magnesium ions is about 10 mM. In some embodiments, the concentration of magnesium ions is about 20 mM. In some embodiments, the concentration of magnesium ions is about 30 mM.

In some embodiments, the washing solution comprises a buffer. The presence of buffers in a solution reduces the magnitude of changes in pH when an acid or base is added to the solution, relative to when the same acid or base is added to a similar composition that does not contain the buffer. Non-limiting examples of buffers for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxy-propanesulfonic acid (MOPSO), N,N-bis-(hydroxyethyl)-2-aminoethanesulfonic acid (BES), 3-(n-morpholino)-propanesulfonic acid (MOPS), N-2-hydroxyethyl-piperazine-N-2-ethanesulfonic acid (HEPES), 3-(N-tris-(hydroxymethyl) methylamino)-2-hydroxypropanesulfonic acid (TAPSO), 3-(N,N-bis [2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl) glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris (hydroxymethyl) aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art. In some embodiments, the washing solution comprises a phosphate buffer. In some embodiments, the washing solution comprises a Tris buffer. In some embodiments, the washing solution comprises an acetate buffer. In some embodiments, the washing solution comprises a histidine buffer. In some embodiments, the washing solution comprises a citrate buffer. Citrate solutions are generally less conductive than other salt solutions such as lithium chloride or sodium acetate solutions. Furthermore, the presence of citrate in a composition comprising RNA can reduce the frequency of base hydrolysis, thereby maintaining the stability of full-length RNA transcripts in the precipitated RNA during washes. In some embodiments, the concentration of the buffer is about 2-10 mM.

In some embodiments, the washing solution comprises an RNase inhibitor. RNase inhibitors limit the activity of RNases. While RNase III described above is useful for cleaving undesired dsRNAs, other RNases cleave single-stranded RNAs, such as RNase T1, which cleaves at guanosine nucleotides, and RNase A, which cleaves at uridine and cytidine residues. Use of RNase inhibitors in a washing solution is therefore useful for inhibiting cleavage of mRNAs in an mRNA composition by contaminating RNases.

In some embodiments, a washing solution comprises EDTA. EDTA refers to ethylenediamine tetraacetic acid, which adsorbs divalent cations (e.g., Mg2+ and Ca2+ ions). Adsorption of divalent cations by EDTA sequesters the cations, and reduces the concentration of those cations in solution. This reduced availability of divalent cations reduces the activity of enzymes that require divalent cations for enzymatic activity, such as RNases. In some embodiments, the concentration of EDTA in a washing solution is about 50 mM to about 1 M. In some embodiments, the concentration of EDTA in a washing solution is about 100 mM to about 500 mM.

In some embodiments, the washing and/or filtration steps are repeated. For example, after a first washing solution is added to a precipitated mRNA composition and removed by filtration, a second washing solution is added and removed by filtration. Each step of (i) adding a washing solution, and (ii) removing the washing solution added in (i) by filtration, is referred to as one “iteration” of the step. Each iteration after the first is referred to as “repeating” a step. Thus, a method in which steps (i) and (ii) are repeated once includes two iterations of each step—a first iteration of step (i), a first iteration of step (ii), a second iteration of step (i), and a second iteration of step (ii). The washing solution added in each iteration of step (i) may be the same washing solution, or a different washing solution. In some embodiments, a washing solution added in one iteration of step (i) comprises the same components (e.g., salt) as a washing solution added in a previous iteration of step (i), but at different concentrations of one or more components. In some embodiments, the same washing solution added in one iteration of step (i) comprises the same components at the same or similar concentrations as a washing solution added in a previous iteration of step (i). In some embodiments, the washing and filtration steps of (i) and (ii) are repeated such that, prior to a resuspension step, the precipitated mRNA has been contacted with a salt, a surfactant, a protease, a DNase, and an RNase III. Adding each of these components to a precipitated mRNA composition allows removal of proteins, organic solvents, proteins, DNA, and dsRNA impurities, respectively. Adding these components during separate washing steps further allows each washing step to target specific impurities, and thus avoid undesired interference between components useful for removing different impurities. For example, the presence of surfactants useful for removing organic solvents or other organic compounds may interfere with the activity of enzymes such as DNase and RNase III. Additionally, washing solutions added in later iterations may contain components useful for removing residual components of washing solutions added in previous iterations. For example, after proteins (e.g., proteases, DNases, RNase III) are added to digest impurities into smaller, more easily filtered fragments, washing solutions containing salts useful for solubilizing and removing proteins may be added in later iterations, enabling removal of pre-existing protein impurities as well as proteins present in previous washing solutions.

In some embodiments, the washing and filtration steps are each performed 2, 3, 4, 5, 6, 7, 8, 9, 10, 11, 12, 13, 14, 15, 16, 17, 18, 19, 20, 21, 22, 23, 24, 25, 26, 27, 28, 29, or 30 times, e.g., prior to a resuspension step. In some embodiments, the washing and filtration steps are performed 2-5, 5-10, 10-15, 15-20, 20-25, or 25-30 times, prior to the resuspension step. In some embodiments, the washing and filtration steps are performed until the concentration of one or more impurities is below a specific amount. In some embodiments, the washing and filtration steps are performed a number of times that is sufficient to reduce the amount of an impurity in the mRNA composition to an undetectable level. In some embodiments, the washing and filtration steps are repeated until the composition is free of detectable protein. In some embodiments, the washing and filtration steps are repeated until the composition is free of detectable DNA. In some embodiments, the washing and filtration steps are repeated until the composition is free of dsRNA. In some embodiments, the washing and filtration steps are repeated until the composition is free of detectable nucleotide triphosphates.

### Resuspension

Some aspects relate to methods of resuspending precipitated mRNA in a solvent with a low concentration of impurities, or an impurity-free solvent, to dissolve the precipitated mRNA and produce a purified mRNA composition. Resuspension and dissolving of precipitated mRNA typically occurs after the salts and/or alcohol used to precipitate the mRNA, and other impurities, are removed by one or more washing steps. Resuspending precipitated mRNA in a solution in which the mRNA is soluble, such as an aqueous buffer with a low salt concentration, results in the precipitated mRNA becoming dissolved in the resuspension solution. Thus, adding a resuspension solution to precipitated mRNA to dissolve the mRNA is also referred to as “resolubilization.”

After salt ions are removed from the precipitated RNA, the conductivity of a liquid, such as a resuspension solution, that is added to precipitated RNA will be lower. The conductivity of a solution comprising resuspended mRNA thus serves as a measure of the solubility of mRNA in the solution. In some embodiments, the resuspension solution, after addition to precipitated RNA, has a conductivity of 10 mS/cm or less, 8 mS/cm or less, 6 mS/cm or less, 5 mS/cm or less, 4 mS/cm or less, 3 mS/cm or less, 2.5 mS/cm or less, 2.0 mS/cm or less, 1.5 mS/cm or less, 1.0 mS/cm or less, or 0.5 mS/cm or less. In some embodiments, the resuspension solution, after addition to precipitated RNA, has a conductivity of 10 mS/cm or less. At low conductivities, the precipitated RNA can be redissolved by adding a resuspension solution. Generally, resuspension solutions contain a low salt concentration, or may contain no salt. In the absence of dissolved salt ions, the phosphates of the precipitated RNA more readily interact with water molecules, allowing the RNA to be dissolved in another solution. In some embodiments, the resuspension solution does not contain detectable salt. In some embodiments, the resuspension solution has a salt concentration of 20 mM or less. In some embodiments, the resuspension solution has a salt concentration of 15 mM or less. In some embodiments, the resuspension solution has a salt concentration of 10 mM or less. In some embodiments, the resuspension solution has a salt concentration of 5 mM or less.

In some embodiments, the resuspension solution comprises a Tris buffer. Tris refers to tris (hydroxymethyl) aminomethane, is an organic compound commonly used in buffers for resuspension and storage of nucleic acids such as DNA and RNA. The buffering activity of Tris reduces the magnitude of pH changes when the composition is contacted with an acid or base, which is useful for preventing undesired effects of hydrogen or hydroxide ions on mRNAs of the composition, such as base hydrolysis. Thus, the presence of Tris inhibits degradation of full-length mRNAs into RNA fragments. Furthermore, in some embodiments, the resuspension solution may have a desired pH, or a pH within a desired range, so that the resuspended mRNA composition has a pH that is suitable for a desired downstream application (e.g., formulation in a delivery vehicle or expression in vivo). In some embodiments, the resuspension solution has a pH of about 5.0 to about 5.5, about 5.5 to about 6.0, about 6.0 to about 6.5, about 6.5 to about 7.0, about 7.0 to about 7.5, about 7.5 to about 8.0, about 6.0 to about 7.0, about 7.0 to about 8.0, or about 6.5 to about 7.5. In some embodiments, the resuspension solution has a pH of about 5.0 to about 8.0. In some embodiments, the resuspension solution has a pH of about 5.0 to about 7.5. In some embodiments, the resuspension solution has a pH of about 5.0 to about 7.0. In some embodiments, the resuspension solution has a pH of about 5.0 to about 6.5. In some embodiments, the resuspension solution has a pH of about 5.0 to about 6.0. In some embodiments, the resuspension solution has a pH of about 5.0 to about 5.2. In some embodiments, the resuspension solution has a pH of about 5.2 to about 5.4. In some embodiments, the resuspension solution has a pH of about 5.4 to about 5.6. In some embodiments, the resuspension solution has a pH of about 5.6 to about 5.8. In some embodiments, the resuspension solution has a pH of about 5.8 to about 6.0. In some embodiments, the resuspension solution has a pH of about 6.0 to about 6.2. In some embodiments, the resuspension solution has a pH of about 6.2 to about 6.4. In some embodiments, the resuspension solution has a pH of about 6.4 to about 6.6. In some embodiments, the resuspension solution has a pH of about 6.6 to about 6.8. In some embodiments, the resuspension solution has a pH of about 6.8 to about 7.0. In some embodiments, the resuspension solution has a pH of about 7.0 to about 7.2. In some embodiments, the resuspension solution has a pH of about 7.2 to about 7.4. In some embodiments, the resuspension solution has a pH of about 7.4 to about 7.6. In some embodiments, the resuspension solution has a pH of about 7.6 to about 7.8. In some embodiments, the resuspension solution has a pH of about 7.8 to about 8.0.

In some embodiments, the resuspension solution comprises an RNase inhibitor. In some embodiments, a resuspension solution comprises EDTA. In some embodiments, the concentration of EDTA in a resuspension solution is about 50 mM to about 1 M. In some embodiments, the concentration of EDTA in a resuspension solution is about 100 mM to about 500 mM.

### Nucleic Acids

Also provided are compositions comprising nucleic acids and methods of producing nucleic acids. As used herein, the term “nucleic acid” includes multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). The term nucleic acid includes polyribonucleotides as well as polydeoxyribonucleotides. The term nucleic acid also includes polynucleosides (i.e., a polynucleotide minus the phosphate) and any other organic base containing polymer. Non-limiting examples of nucleic acids include chromosomes, genomic loci, genes, or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleotides having an organic group, such as a methyl group, attached to a nucleic acid base at the N6 position. Thus, in some embodiments, an mRNA includes one or more N6-methyladenosine nucleotides. A phosphate, sugar, or nucleic acid base of a nucleotide may also be substituted for another phosphate, sugar, or nucleic acid base. For example, a uridine base may be substituted for a pseudouridine base, in which the uracil base is attached to the sugar by a carbon-carbon bond rather than a nitrogen-carbon bond. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acid sequences provided herein include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence.

Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids, or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

In some embodiments, a nucleic acid is present in (or on) a vector. Examples of vectors include but are not limited to bacterial plasmids, phage, cosmids, phasmids, fosmids, bacterial artificial chromosomes, yeast artificial chromosomes, viruses, and retroviruses (for example vaccinia, adenovirus, adeno-associated virus, lentivirus, herpes-simplex virus, Epstein-Barr virus, fowlpox virus, pseudorabies, baculovirus) and vectors derived therefrom. In some embodiments, a nucleic acid (e.g., DNA) used as an input molecule for in vitro transcription (IVT) is present in a plasmid vector.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

Aspects of the disclosure relate to populations of molecules. As used herein, a “population” of molecules (e.g., DNA molecules) generally refers to a preparation (e.g., a plasmid preparation) comprising a plurality of copies of the molecule (e.g., DNA) of interest, for example a cell extract preparation comprising a plurality of expression vectors encoding a molecule of interest (e.g., a DNA encoding an RNA of interest). In some embodiments, a population is a homogenous population comprising a single RNA species. As used herein, an RNA species refers to an RNA molecule having a given nucleotide sequence. Two or more RNA molecules having identical nucleotide sequences and backbone compositions belong to the same RNA species, while two RNA molecules having different nucleotide sequences and/or different backbone compositions belong to different RNA species. In some embodiments, a population a heterogenous population comprising two or more RNA species. In some embodiments, a heterogenous population comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, or more RNA species.

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Examples of naturally-occurring nucleotides used for the production of RNA, e.g., in an IVT reaction, as provided herein include adenosine triphosphate (ATP), guanosine triphosphate (GTP), cytidine triphosphate (CTP), uridine triphosphate (UTP), and 5-methyluridine triphosphate (m5UTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO₄ to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, an RNA transcript (e.g., mRNA transcript) provided herein may include a modified nucleobase selected from pseudouridine (v), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, an RNA transcript (e.g., mRNA transcript) includes a combination of at least two (e.g., 2, 3, 4 or more) of the foregoing modified nucleobases.

Some aspects relate to mRNAs produced by “in vitro transcription” or IVT. IVT methods produce (e.g., synthesize) an RNA transcript (e.g., mRNA transcript) by contacting a DNA template (e.g., a first input DNA and a second input DNA) with an RNA polymerase (e.g., a T7 RNA polymerase, a T7 RNA polymerase variant, etc.) under conditions that result in the production of the RNA transcript. IVT conditions typically require a purified DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

In some embodiments, methods described herein further comprise a step of separating (e.g., purifying) in vitro transcription products (e.g., mRNA) from other reaction components. In some embodiments, the separating comprises performing chromatography on the mRNA composition. In some embodiments, the method comprises reverse phase chromatography. In some embodiments, the method comprises reverse phase column chromatography. In some embodiments, the chromatography comprises size-based (e.g., length-based) chromatography. In some embodiments, the method comprises size exclusion chromatography. In some embodiments, the chromatography comprises oligo-dT chromatography. mRNAs purified by the methods described herein and/or present in the mRNA compositions described herein may encode, in some embodiments, a vaccine antigen or therapeutic protein. As used herein, a “vaccine antigen” is a biological preparation that improves immunity to a particular disease or infectious agent. Vaccine antigens encoded by an mRNA described herein may be utilized to treat conditions or diseases in many therapeutic areas such as, but not limited to, cancer, allergy and infectious disease. In some embodiments the cancer vaccines may be personalized cancer vaccines in the form of a concatemer or individual RNAs encoding peptide epitopes or a combination thereof.

The mRNAs described herein may be designed to encode on or more antimicrobial peptides (AMP) or antiviral peptides (AVP). AMPs and AVPs have been isolated and described from a wide range of animals such as, but not limited to, microorganisms, invertebrates, plants, amphibians, birds, fish, and mammals. The anti-microbial polypeptides described herein may block cell fusion and/or viral entry by one or more enveloped viruses (e.g., HIV, HCV). For example, the anti-microbial polypeptide can comprise or consist of a synthetic peptide corresponding to a region, e.g., a consecutive sequence of at least about 5, 10, 15, 20, 25, 30, 35, 40, 45, 50, 55, or 60 amino acids of the transmembrane subunit of a viral envelope protein, e.g., HIV-1 gp120 or gp41. The amino acid and nucleotide sequences of HIV-1 gp120 or gp41 are described in, e.g., Kuiken et al., (2008). “HIV Sequence Compendium,” Los Alamos National Laboratory.

In some embodiments, the anti-microbial polypeptide may have at least about 75%, 80%, 85%, 90%, 95%, 100% sequence homology to the corresponding viral protein sequence. In some embodiments, the anti-microbial polypeptide may have at least about 75%, 80%, 85%, 90%, 95%, or 100% sequence homology to the corresponding viral protein sequence.

In other embodiments, the anti-microbial polypeptide may comprise or consist of a synthetic peptide corresponding to a region, e.g., a consecutive sequence of at least about 5, 10, 15, 20, 25, 30, 35, 40, 45, 50, 55, or 60 amino acids of the binding domain of a capsid binding protein. In some embodiments, the anti-microbial polypeptide may have at least about 75%, 80%, 85%, 90%, 95%, or 100% sequence homology to the corresponding sequence of the capsid binding protein.

The anti-microbial polypeptides described herein may block protease dimerization and inhibit cleavage of viral proproteins (e.g., HIV Gag-pol processing) into functional proteins thereby preventing release of one or more enveloped viruses (e.g., HIV, HCV). In some embodiments, the anti-microbial polypeptide may have at least about 75%, 80%, 85%, 90%, 95%, 100% sequence homology to the corresponding viral protein sequence.

In other embodiments, the anti-microbial polypeptide can comprise or consist of a synthetic peptide corresponding to a region, e.g., a consecutive sequence of at least about 5, 10, 15, 20, 25, 30, 35, 40, 45, 50, 55, or 60 amino acids of the binding domain of a protease binding protein. In some embodiments, the anti-microbial polypeptide may have at least about 75%, 80%, 85%, 90%, 95%, 100% sequence homology to the corresponding sequence of the protease binding protein.

A non-limiting list of infectious diseases that the RNA vaccine antigens or anti-microbial peptides may treat is presented below: human immunodeficiency virus (HIV), HIV resulting in mycobacterial infection, AIDS related Cacheixa, AIDS related Cytomegalovirus infection, HIV-associated nephropathy, Lipodystrophy, AID related cryptococcal meningitis, AIDS-related neutropenia, Pneumocystis carinii infections, AID related toxoplasmosis, hepatitis A, B, C, D or E, herpes, herpes zoster (chicken pox), German measles (rubella virus), yellow fever, dengue fever etc. (flavi viruses), flu (influenza viruses), hemorrhagic infectious diseases (Marburg or Ebola viruses), bacterial infectious diseases such as Legionnaires' disease (Legionella), gastric ulcer (Helicobacter), cholera (Vibrio), E. coli infections, staphylococcal infections, salmonella infections or streptococcal infections, tetanus (Clostridium tetani), protozoan infectious diseases (malaria, sleeping sickness, leishmaniasis, toxoplasmosis, i.e. infections caused by plasmodium, trypanosomes, leishmania and toxoplasma), diphtheria, leprosy, measles, pertussis, rabies, tetanus, tuberculosis, typhoid, varicella, diarrheal infections such as Amoebiasis, Clostridium difficile-associated diarrhea (CDAD), Cryptosporidiosis, Giardiasis, Cyclosporiasis and Rotaviral gastroenteritis, encephalitis such as Japanese encephalitis, Wester equine encephalitis and Tick-borne encephalitis (TBE), fungal skin diseases such as candidiasis, onychomycosis, Tinea captis/scal ringworm, Tinea corporis/body ringworm, Tinea cruris/jock itch, sporotrichosis and Tinea pedis/Athlete's foot, Meningitis such as Haemophilus influenza type b (Hib), Meningitis, viral, meningococcal infections and pneumococcal infection, neglected tropical diseases such as Argentine haemorrhagic fever, Leishmaniasis, Nematode/roundworm infections, Ross river virus infection and West Nile virus (WNV) disease, Non-HIV STDs such as Trichomoniasis, Human papillomavirus (HPV) infections, sexually transmitted chlamydial diseases, Chancroid and Syphilis, Non-septic bacterial infections such as cellulitis, lyme disease, MRSA infection, pseudomonas, staphylococcal infections, Boutonneuse fever, Leptospirosis, Rheumatic fever, Botulism, Rickettsial disease and Mastoiditis, parasitic infections such as Cysticercosis, Echinococcosis, Trematode/Fluke infections, Trichinellosis, Babesiosis, Hypodermyiasis, Diphyllobothriasis and Trypanosomiasis, respiratory infections such as adenovirus infection, aspergillosis infections, avian (H5N1) influenza, influenza, RSV infections, severe acute respiratory syndrome (SARS), sinusitis, Legionellosis, Coccidioidomycosis and swine (H1N1) influenza, sepsis such as bacteraemia, sepsis/septic shock, sepsis in premature infants, urinary tract infection such as vaginal infections (bacterial), vaginal infections (fungal) and gonococcal infection, viral skin diseases such as B19 parvovirus infections, warts, genital herpes, orofacial herpes, shingles, inner ear infections, fetal cytomegalovirus syndrome, foodborn illnesses such as brucellosis (Brucella species), Clostridium perfringens (Epsilon toxin), E. coli O157: H7 (Escherichia coli), Salmonellosis (Salmonella species), Shingellosis (Shingella), Vibriosis and Listeriosis, bioterrorism and potential epidemic diseases such as Ebola haemorrhagic fever, Lassa fever, Marburg haemorrhagic fever, plague, Anthrax Nipah virus disease, Hanta virus, Smallpox, Glanders (Burkholderia mallei), Melioidosis (Burkholderia pseudomallei), Psittacosis (Chlamydia psittaci), Q fever (Coxiella burnetii), Tularemia (Fancisella tularensis), rubella, mumps and polio.

The RNA disclosed herein, may encode one or more validated or “in testing” therapeutic proteins or peptides. In some embodiments, one or more therapeutic proteins or peptides currently being marketed or in development may be encoded by an mRNA of the compositions and methods described herein. Therapeutic proteins and peptides encoded by the mRNA may be utilized to treat conditions or diseases in many therapeutic areas such as, but not limited to, blood, cardiovascular, CNS, poisoning (including antivenoms), dermatology, endocrinology, genetic, genitourinary, gastrointestinal, musculoskeletal, oncology, and immunology, respiratory, sensory, and anti-infective applications.

The mRNAs of the compositions and methods described herein may encode one or more cell-penetrating polypeptides. As used herein, “cell-penetrating polypeptide” or CPP refers to a polypeptide which may facilitate the cellular uptake of molecules. A cell-penetrating polypeptide may contain one or more detectable labels. The polypeptides may be partially labeled or completely labeled throughout. The RNA may encode the detectable label completely, partially or not at all. The cell-penetrating peptide may also include a signal sequence. As used herein, a “signal sequence” refers to a sequence of amino acid residues bound at the amino terminus of a nascent protein during protein translation. The signal sequence may be used to signal the secretion of the cell-penetrating polypeptide.

In one embodiment, the RNA may also encode a fusion protein. The fusion protein may be created by operably linking a charged protein to a therapeutic protein. As used herein, “operably linked” refers to the therapeutic protein and the charged protein being connected in such a way to permit the expression of the complex when introduced into the cell. As used herein, “charged protein” refers to a protein that carries a positive, negative or overall neutral electrical charge. Preferably, the therapeutic protein may be covalently linked to the charged protein in the formation of the fusion protein. The ratio of surface charge to total or surface amino acids may be approximately 0.1, 0.2, 0.3, 0.4, 0.5, 0.6, 0.7, 0.8 or 0.9.

The cell-penetrating polypeptide encoded by the RNA may form a complex after being translated. The complex may comprise a charged protein linked, e.g. covalently linked, to the cell-penetrating polypeptide.

In one embodiment, the cell-penetrating polypeptide may comprise a first domain and a second domain. The first domain may comprise a supercharged polypeptide. The second domain may comprise a protein-binding partner. As used herein, “protein-binding partner” includes, but is not limited to, antibodies and functional fragments thereof, scaffold proteins, or peptides. The cell-penetrating polypeptide may further comprise an intracellular binding partner for the protein-binding partner. The cell-penetrating polypeptide may be capable of being secreted from a cell where the RNA may be introduced. The cell-penetrating polypeptide may also be capable of penetrating the first cell.

In one embodiment, the RNA may encode a cell-penetrating polypeptide which may comprise a protein-binding partner. The protein binding partner may include, but is not limited to, an antibody, a supercharged antibody or a functional fragment. The RNA may be introduced into the cell where a cell-penetrating polypeptide comprising the protein-binding partner is introduced.

Human and other eukaryotic cells are subdivided by membranes into many functionally distinct compartments. Each membrane-bound compartment, or organelle, contains different proteins essential for the function of the organelle. The cell uses “sorting signals” which are amino acid motifs located within the protein, to target proteins to particular cellular organelles. One type of sorting signal, called a signal sequence, a signal peptide, or a leader sequence, directs a class of proteins to an organelle called the endoplasmic reticulum (ER). Proteins targeted to the ER by a signal sequence can be released into the extracellular space as a secreted protein. Similarly, proteins residing on the cell membrane can also be secreted into the extracellular space by proteolytic cleavage of a “linker” holding the protein to the membrane. While not wishing to be bound by theory, the molecules may be used to exploit the cellular trafficking described above. As such, in some embodiments, an mRNA is express a secreted protein. In one embodiment, these may be used in the manufacture of large quantities of valuable human gene products.

In some embodiments, an mRNA can express a protein of the plasma membrane. In some embodiments, an mRNA can express a cytoplasmic or cytoskeletal protein. In some embodiments, an mRNA can express an intracellular membrane bound protein. In some embodiments, an mRNA can express a nuclear protein. In some embodiments, an mRNA can express a protein associated with human disease.

### Untranslated Regions

Untranslated regions (UTRs) are sections of a nucleic acid before a start codon (5′ UTR) and after a stop codon (3′ UTR) that are not translated. In some embodiments, a nucleic acid (e.g., a ribonucleic acid (RNA), e.g., a messenger RNA (mRNA)) of the disclosure comprising an open reading frame (ORF) encoding one or more proteins or peptides further comprises one or more UTR (e.g., a 5′ UTR or functional fragment thereof, a 3′ UTR or functional fragment thereof, or a combination thereof).

A UTR can be homologous or heterologous to the coding region in a nucleic acid. In some embodiments, the UTR is homologous to the ORF encoding the one or more peptide epitopes. In some embodiments, the UTR is heterologous to the ORF encoding the one or more peptide epitopes. In some embodiments, the nucleic acid comprises two or more 5′ UTRs or functional fragments thereof, each of which have the same or different nucleotide sequences. In some embodiments, the nucleic acid comprises two or more 3′ UTRs or functional fragments thereof, each of which have the same or different nucleotide sequences.

In some embodiments, the 5′ UTR or functional fragment thereof, 3′ UTR or functional fragment thereof, or any combination thereof is sequence optimized.

In some embodiments, the 5′ UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization, and/or translation efficiency. A nucleic acid comprising a UTR can be administered to a cell, tissue, or organism, and one or more regulatory features can be measured using routine methods. In some embodiments, a functional fragment of a 5′ UTR or 3′ UTR comprises one or more regulatory features of a full length 5′ or 3′ UTR, respectively.

Natural 5′ UTRs bear features that play roles in translation initiation. They harbor signatures like Kozak sequences that are commonly known to be involved in the process by which the ribosome initiates translation of many genes. 5′ UTRs also have been known to form secondary structures that are involved in elongation factor binding.

By engineering the features typically found in abundantly expressed genes of specific target organs, one can enhance the stability and protein production of a nucleic acid. For example, introduction of 5′ UTR of liver-expressed mRNA, such as albumin, serum amyloid A, Apolipoprotein A/B/E, transferrin, alpha fetoprotein, erythropoietin, or Factor VIII, can enhance expression of nucleic acids in hepatic cell lines or liver. Likewise, use of 5′ UTRs from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin), and for lung epithelial cells (e.g., SP-A/B/C/D).

In some embodiments, UTRs are selected from a family of transcripts whose proteins share a common function, structure, feature, or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new nucleic acid. In some embodiments, the 5′ UTR and the 3′ UTR can be heterologous. In some embodiments, the 5′ UTR can be derived from a different species than the 3′ UTR. In some embodiments, the 3′ UTR can be derived from a different species than the 5′ UTR.

International Patent Application No. PCT/US2014/021522 (Publ. No. WO/2014/164253) provides a listing of exemplary UTRs that may be utilized in the nucleic acids provided herein as flanking regions to an ORF. This publication is incorporated by reference herein for this purpose.

Additional exemplary UTRs that may be utilized in the nucleic acids provided herein include, but are not limited to, one or more 5′ UTRs and/or 3′ UTRs derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 a polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV; e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′ UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1(Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

In some embodiments, the 5′ UTR is selected from the group consisting of a β-globin 5′ UTR; a 5′ UTR containing a strong Kozak translational initiation signal; a cytochrome b-245 α polypeptide (CYBA) 5′ UTR; a hydroxysteroid (17-β) dehydrogenase (HSD17B4) 5′ UTR; a Tobacco etch virus (TEV) 5′ UTR; a Venezuelen equine encephalitis virus (TEEV) 5′ UTR; a 5′ proximal open reading frame of rubella virus (RV) RNA encoding nonstructural proteins; a Dengue virus (DEN) 5′ UTR; a heat shock protein 70 (Hsp70) 5′ UTR; a eIF4G 5′ UTR; a GLUT1 5′ UTR; functional fragments thereof and any combination thereof.

In some embodiments, the 3′ UTR is selected from the group consisting of a β-globin 3′ UTR; a CYBA 3′ UTR; an albumin 3′ UTR; a growth hormone (GH) 3′ UTR; a VEEV 3′ UTR; a hepatitis B virus (HBV) 3′ UTR; α-globin 3′ UTR; a DEN 3′ UTR; a PAV barley yellow dwarf virus (BYDV-PAV) 3′ UTR; an elongation factor 1 α1 (EEF1A1) 3′ UTR; a manganese superoxide dismutase (MnSOD) 3′ UTR; a β subunit of mitochondrial H (+)-ATP synthase (β-mRNA) 3′ UTR; a GLUT1 3′ UTR; a MEF2A 3′ UTR; a β-F1-ATPase 3′ UTR; functional fragments thereof and combinations thereof.

Wild-type UTRs derived from any gene or mRNA can be incorporated into the nucleic acids of the disclosure. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8 (3): 568-82, and sequences available at www.addgene.org, the contents of each are incorporated herein by reference in their entirety. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the nucleic acid may comprise multiple UTRs, e.g., a double, a triple or a quadruple 5′ UTR or 3′ UTR. For example, a double UTR comprises two copies of the same UTR either in series or substantially in series. For example, a double beta-globin 3′ UTR can be used (see, for example, US2010/0129877, the contents of which are incorporated herein by reference for this purpose).

The nucleic acids of the disclosure can comprise combinations of features. For example, the ORF can be flanked by a 5′ UTR that comprises a strong Kozak translational initiation signal and/or a 3′ UTR comprising an oligo (dT) sequence for templated addition of a polyA tail. A 5′ UTR can comprise a first nucleic acid fragment and a second nucleic acid fragment from the same and/or different UTRs (see, e.g., US2010/0293625, herein incorporated by reference in its entirety for this purpose).

Other non-UTR sequences can be used as regions or subregions within the nucleic acids of the disclosure. For example, introns or portions of intron sequences can be incorporated into the nucleic acids of the disclosure. Incorporation of intronic sequences can increase protein production as well as nucleic acid expression levels. In some embodiments, the nucleic acid of the disclosure comprises an internal ribosome entry site (IRES) instead of or in addition to a UTR (see, e.g., Yakubov et al., Biochem. Biophys. Res. Commun. 2010 394 (1): 189-193, the contents of which are incorporated herein by reference in their entirety). In some embodiments, the nucleic acid comprises an IRES instead of a 5′ UTR sequence. In some embodiments, the nucleic acid comprises an IRES that is located between a 5′ UTR and an open reading frame. In some embodiments, the nucleic acid comprises an ORF encoding a viral capsid sequence. In some embodiments, the nucleic acid comprises a synthetic 5′ UTR in combination with a non-synthetic 3′ UTR.

In some embodiments, the UTR can also include at least one translation enhancer nucleic acid, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety for this purpose, and others known in the art. As a non-limiting example, the TEE can be located between the transcription promoter and the start codon. In some embodiments, the 5′ UTR comprises a TEE. In one aspect, a TEE is a conserved element in a UTR that can promote translational activity of a nucleic acid such as, but not limited to, cap-dependent or cap-independent translation. In one non-limiting example, the TEE comprises the TEE sequence in the 5′-leader of the Gtx homeodomain protein. See, e.g., Chappell et al., PNAS. 2004. 101:9590-9594, incorporated herein by reference in its entirety for this purpose.

### Poly(A) Tails

Some aspects relate to methods of producing RNAs containing one or more polyA tails. A “polyA tail” is a region of mRNA that is downstream, e.g., directly downstream (i.e., 3′), from the open reading frame and/or the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly (A) tail functions to protect mRNA from enzymatic degradation, e.g., in the cytoplasm, and aids in transcription termination, export of the mRNA from the nucleus, and translation.

As used herein, “polyA-tailing efficiency” refers to the amount (e.g., expressed as a percentage) of mRNAs having polyA tail that are produced by an IVT reaction using an input DNA relative to the total number of mRNAs produced in the IVT reaction using the input DNA. The poly A-tailing efficiency of an IVT reaction may vary, for example depending upon the RNA polymerase used, amount or purity of input DNA used, etc. In some embodiments, the polyA-tailing efficiency of an IVT reaction is greater than 85%, 90%, 95%, or 99.9%. Methods of calculating polyA-tailing efficiency are known, for example by determining the amount of polyA tail-containing mRNA relative to total mRNA produced in an IVT reaction by column chromatography (e.g., oligo-dT chromatography).

In some embodiments, at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.9% of RNAs in an RNA composition produced by a method described herein comprise a polyA tail. In some embodiments, at least 85%, 86%, 87%, 88%, 89%, 90%, 91%, 92%, 93%, 94%, 95%, 96%, 97%, 98%, 99%, or 99.9% of each RNA in an RNA composition produced by a method described herein comprise a polyA tail. The efficiency (e.g., percentage of polyA tail-containing

RNAs in an RNA composition may be measured i) after the IVT reaction and before purification, or ii) after the RNA composition has been purified (e.g., by chromatography, such as oligo-dT chromatography).

Unique polyA tail lengths provide certain advantages to the nucleic acids provided herein. Generally, the length of a polyA tail, when present, is greater than 30 nucleotides in length. In another embodiment, the polyA tail is greater than 35 nucleotides in length (e.g., at least or greater than about 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides).

In some embodiments, the polyA tail is designed relative to the length of the overall nucleic acid or the length of a particular region of the nucleic acid. This design can be based on the length of a coding region, the length of a particular feature or region or based on the length of the ultimate product expressed from the nucleic acids.

In this context, the polyA tail can be 10, 20, 30, 40, 50, 60, 70, 80, 90, or 100% greater in length than the nucleic acid or feature thereof. The polyA tail can also be designed as a fraction of the nucleic acid to which it belongs. In this context, the polyA tail can be 10, 20, 30, 40, 50, 60, 70, 80, or 90% or more of the total length of the construct, a construct region, or the total length of the construct minus the polyA tail. Further, engineered binding sites and conjugation of nucleic acids for PolyA-binding protein can enhance expression.

### mRNA Compositions

Aspects of the disclosure relate to compositions comprising mRNA purified by any of the methods described herein. In some embodiments, mRNA compositions produced by the methods described herein are more pure than mRNA compositions purified by alternative methods, such as oligo-dT or high-performance liquid chromatography. Whether a composition is more pure than a composition produced by an alternative method may be determined by methods known in the art, including separating a composition to be purified into multiple equivalent samples, purifying each by a different method, and comparing the contents of the resulting purified composition. A first mRNA composition comprising a lower abundance or concentration of a given impurity than a second mRNA composition is said to be “more pure” than the second mRNA composition. When the first and second purified mRNA compositions are produced by purifying similar samples of an initial mRNA composition by a first and second method, and the first purified mRNA composition has a lower concentration of an impurity than the second purified mRNA composition, the first method is said to be more effective at removing the impurity than the second method.

In some embodiments, the purified mRNA composition produced by a method described herein comprises at least 80% of the mRNA that was present in the mRNA that was present before precipitation. In some embodiments, the purified mRNA composition comprises at least 85% of the mRNA that was present in the composition before precipitation. In some embodiments, the purified mRNA composition comprises at least 90% of the mRNA that was present in the composition before precipitation. In some embodiments, the purified mRNA composition comprises at least 95% of the mRNA that was present in the composition before precipitation. In some embodiments, the purified mRNA composition comprises at least 96% of the mRNA that was present in the composition before precipitation. In some embodiments, the purified mRNA composition comprises at least 97% of the mRNA that was present in the composition before precipitation. In some embodiments, the purified mRNA composition comprises at least 98% of the mRNA that was present in the composition before precipitation. In some embodiments, the purified mRNA composition comprises at least 99% of the mRNA that was present in the composition before precipitation. In some embodiments, the purified mRNA composition comprises up to 100% of the mRNA that was present in the composition before precipitation.

In some embodiments, the purified mRNA composition produced by a method described herein has a dsRNA concentration that is lower than the dsRNA concentration of a purified mRNA composition produced by purifying a similar initial mRNA composition by chromatography. Methods of measuring the presence and/or amount of dsRNA in a composition are known in the art. Non-limiting examples of methods for measuring dsRNA content of a sample include ELISAs and immunoblotting using antibodies specific to dsRNA. Additionally, the total mass of RNA in a sample can be measured using techniques such as spectroscopy (NanoDrop), qRT-PCR, and/or ddPCR, and the mass of dsRNA can be measured using an intercalating agent that fluoresces when bound to dsRNA, such as acridine orange, with the dsRNA concentration being calculated by division. In some embodiments, the concentration of double-stranded RNA in a composition comprising RNA is 5% (% w/w) or less, 4% or less, 3% or less, 2.5% or less, 2% or less, 1.75% or less, 1.5% or less, 1.25% or less, 1% or less, 0.9% or less, 0.8% or less, 0.7% or less, 0.6% or less, 0.5% or less, 0.4% or less, 0.3% or less, 0.25% or less, 0.2% or less, 0.175% or less, 0.15% or less, 0.125% or less, or 0.1% or less. In some embodiments, the concentration of double-stranded RNA in a composition comprising RNA is 1% (% w/w) or less, 0.9% or less, 0.8% or less, 0.7% or less, 0.6% or less, 0.5% or less, 0.4% or less, 0.3% or less, 0.2% or less, or 0.1% or less. In some embodiments, the concentration of double-stranded RNA in a composition comprising RNA is 0.05% (% w/w) or less, 0.04% or less, 0.03% or less, 0.02% or less, or 0.01% or less.

In some embodiments, the percentage of RNAs that are dsRNAs in a purified composition produced by a method described herein is lower than the percentage of dsRNAs in a purified mRNA composition produced by purifying a similar initial mRNA composition by chromatography. In some embodiments, 1% or fewer of RNAs in a composition are dsRNAs. In some embodiments, 0.9% or fewer of RNAs in a composition are dsRNAs. In some embodiments, 0.8% or fewer of RNAs in a composition are dsRNAs. In some embodiments, 0.7% or fewer of RNAs in a composition are dsRNAs. In some embodiments, 0.6% or fewer of RNAs in a composition are dsRNAs. In some embodiments, 0.5% or fewer of RNAs in a composition are dsRNAs. In some embodiments, 0.4% or fewer of RNAs in a composition are dsRNAs. In some embodiments, 0.3% or fewer of RNAs in a composition are dsRNAs. In some embodiments, 0.2% or fewer of RNAs in a composition are dsRNAs. In some embodiments, 0.1% or fewer of RNAs in a composition are dsRNAs. In some embodiments, 0.05% or fewer of RNAs in a composition are dsRNAs. In some embodiments, 0.04% or fewer of RNAs in a composition are dsRNAs. In some embodiments, 0.03% or fewer of RNAs in a composition are dsRNAs. In some embodiments, 0.02% or fewer of RNAs in a composition are dsRNAs. In some embodiments, 0.0% or fewer of RNAs in a composition are dsRNAs.

In some embodiments, the purified mRNA composition produced by a method described herein has a protein concentration that is lower than the protein concentration of a purified mRNA composition produced by purifying a similar initial mRNA composition by chromatography. In some embodiments, the concentration of proteins in the RNA composition produced by the method is 1.0% (% w/w) or less, 0.8% or less, 0.6% or less, 0.4% or less, or 0.2% or less. Methods of measuring the amount of protein in a sample include colorimetric assays (e.g., Bradford assay), spectroscopy, ELISA, polyacrylamide gel electrophoresis electropherogram analysis, and chromatographic analysis. See, e.g., Sapan et al., Biotechnol Appl Biochem. 1999. 29(2): 99-108. In some embodiments, the concentration of proteins in the RNA composition is 1.0% or less. In some embodiments, the concentration of proteins in the RNA composition is 0.9% or less. In some embodiments, the concentration of proteins in the RNA composition is 0.8% or less. In some embodiments, the concentration of proteins in the RNA composition is 0.6% or less. In some embodiments, the concentration of proteins in the RNA composition is 0.5% or less. In some embodiments, the concentration of proteins in the RNA composition is 0.4% or less. In some embodiments, the concentration of proteins in the RNA composition is 0.3% or less. In some embodiments, the concentration of proteins in the RNA composition is 0.2% or less.

In some embodiments, the purified mRNA composition produced by a method described herein has a DNA concentration that is lower than the DNA concentration of a purified mRNA composition produced by purifying a similar initial mRNA composition by chromatography. Methods of measuring the presence and/or amount of DNA in a composition are known in the art. Non-limiting examples of methods for measuring dsRNA content of a sample include gel electrophoresis using an intercalating agent that fluoresces when bound to DNA (e.g., ethidium bromide), spectroscopy (NanoDrop), qPCR, and/or ddPCR. In some embodiments, the concentration of DNA in the RNA composition is 1.0% or less. In some embodiments, the concentration of DNA in the RNA composition is 0.9% or less. In some embodiments, the concentration of DNA in the RNA composition is 0.8% or less. In some embodiments, the concentration of DNA in the RNA composition is 0.6% or less. In some embodiments, the concentration of DNA in the RNA composition is 0.5% or less. In some embodiments, the concentration of DNA in the RNA composition is 0.4% or less. In some embodiments, the concentration of DNA in the RNA composition is 0.3% or less. In some embodiments, the concentration of DNA in the RNA composition is 0.2% or less.

### EXAMPLES

### Example 1: Precipitation for mRNA Capture and Impurity Removal by Washing

mRNA compositions containing impurities are purified by (i) precipitating mRNA; (ii) removing the aqueous phase containing soluble impurities, or a portion thereof; (iii)(a) washing the precipitated mRNA by adding a washing solution; (iii)(b) removing the washing solution, or a portion thereof; and (iv) resuspending the precipitated mRNA. The methods described in this Example may be used to purify mRNAs produced by a cell-free process, such as in vitro transcription, mRNAs isolated from cells, or mRNAs obtained from a commercial supplier.

A solution containing an appropriate salt in a concentration sufficient to bring the composition to a desired concentration of the salt is mixed with an mRNA composition to promote nucleation and aggregation and precipitation of mRNAs. After salt addition, a volume exclusion agent, such as an alcohol (e.g., ethanol or isopropanol) is added to promote further aggregation of the mRNAs, increasing the rate of precipitation. Nucleic acids are less soluble in alcohols, such as isopropanol, and so the addition of an alcohol promotes interactions between mRNAs in the aqueous portion of the composition. Precipitation is performed at room temperature, to reduce the amount of solutes (e.g., sucrose or sodium chloride) that are coprecipitated with the mRNA. During precipitation, the turbidity, osmolality, and particle size distribution of the mRNA composition are monitored in-line using dynamic light scattering and micro-flow imaging, to evaluate the extent of precipitation over time.

After precipitation, the aqueous phase containing dissolved impurities is separated by filtration using a hollow fiber membrane having a 0.2 μm pore size. This step removes many soluble impurities, and remaining impurities are then removed by washing the precipitate with an alcohol solution (e.g., 70% ethanol or isopropanol), and passing the washing solution: precipitate mixture over a tangential flow filtration (TFF) membrane with a molecular weight cutoff of 30 kDa to remove the washing solution containing dissolved impurities. This wash step is then repeated, which acts to further dilute and remove remaining impurities, before the precipitated mRNA is redissolved. Compared to using an equivalent total volume of washing solution in a single wash step, serially diluting and removing impurities in this manner increases the efficiency of purification.

After impurities are removed by washing, a resuspension solution of 10 mM Tris-HCl pH 5.5 is added to the precipitate to dissolve the mRNA. The resulting mRNA composition is then analyzed to determine the presence of protein, DNA, and dsRNA impurities. The amount of mRNA in the resuspended mRNA composition is quantified by HPLC or spectrometry, to determine the mRNA concentration for use in downstream applications. This mRNA amount is then compared to the amount of mRNA present in the mRNA composition before precipitation, to determine the percent yield of the purification process. mRNA purity (e.g., percentage of mRNAs having expected size and/or containing a poly(A) tail) is also measured, such as by HPLC, to confirm the suitability of mRNA for downstream applications.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A continuous method of removing one or more impurities from a composition comprising messenger ribonucleic acid (mRNA), the continuous method comprising:
(a) precipitating mRNA from the composition to form a precipitated mRNA composition;
(b) removing one or more impurities from the precipitated mRNA composition; and then
(c) resuspending the precipitated mRNA.

2. The continuous method of claim 1, wherein the precipitating comprises adding a high-salt buffer and/or alcohol to the composition comprising the mRNA to form the precipitated mRNA composition.

3. The continuous method of claim 1 or 2, wherein the removing one or more impurities comprises contacting the precipitated mRNA composition with a filter membrane.

4. The continuous method of any one of claims 1-3, wherein the removing one or more impurities comprises contacting the precipitated mRNA composition with a washing solution.

5. The continuous method of any one of claims 1-4, wherein the removing one or more impurities comprises contacting the precipitated mRNA composition with a second filter membrane.

6. The continuous method of any one of claims 1-5, wherein the removing one or more impurities comprises contacting the precipitated mRNA composition with a second washing solution.

7. A continuous method of any one of claims 1-6, the method comprising:
(i) adding a high-salt buffer to the mRNA composition to form a precipitated mRNA composition;
(ii) contacting the precipitated mRNA composition with a filter membrane to remove one or more impurities from the precipitated mRNA composition;
(iii)(a) contacting the precipitated mRNA composition with a washing solution;
(iii)(b) contacting the precipitated mRNA composition with a second filter membrane to remove one or more impurities from the precipitated mRNA composition; and
(iv) contacting the precipitated mRNA composition with a resuspension solution to resuspend the mRNA.

8. The method of claim 2 or 7, wherein the high-salt buffer comprises a salt selected from the group consisting of lithium chloride, lithium acetate, lithium sulfate, sodium chloride, sodium acetate, sodium sulfate, ammonium chloride, ammonium acetate, and ammonium sulfate.

9. The method of any one of claim 2 or 7-8, wherein the high-salt buffer comprises ammonium sulfate.

10. The method of any one of claim 2 or 7-9, wherein the high-salt buffer has a salt concentration of about 0.1 M to about 5.0 M.

11. The method of any one of claim 2 or 7-10, wherein the high-salt buffer has a conductivity of 5 mS/cm or more, optionally wherein the high-salt buffer has a conductivity of about 5 mS/cm to about 300 mS/cm.

12. The method of any one of claims 1-11, wherein the precipitation step further comprises adding an alcohol to the mRNA composition.

13. The method of claim 12, wherein the alcohol is ethanol or isopropanol.

14. The method of any one of claim 3 or 7-13, wherein the filter membrane is a tangential flow filtration (TFF) membrane.

15. The method of any one of claim 3 or 7-14, wherein the filter membrane is a hollow fiber membrane.

16. The method of any one of claim 3 or 7-15, wherein the filter membrane has a molecular weight cutoff of about 20 kDa to about 150 kDa.

17. The method of any one of claim 4 or 7-16, wherein the washing solution comprises a surfactant, a salt, a DNase, and/or an RNase III.

18. The method of any one of claim 4 or 7-17, wherein the washing solution comprises a surfactant.

19. The method of claim 18, wherein the washing solution comprises a detergent.

20. The method of claim 19, wherein the detergent is a Triton X-100 detergent.

21. The method of any one of claim 4 or 7-20, wherein the washing solution comprises a salt.

22. The method of claim 21, wherein the salt comprises a monovalent or divalent cation.

23. The method of claim 21, wherein the monovalent or divalent cation is selected from the group consisting of lithium, sodium, ammonium, calcium, and magnesium.

24. The method of any one of claims 21-23, wherein comprises an anion selected from the group consisting of chloride, sulfate, and acetate.

25. The method of any one of claim 4 or 7-24, wherein the washing solution comprises sodium chloride, calcium chloride, and/or ammonium sulfate.

26. The method of any one of claims 21-25, wherein the salt concentration in the washing solution is between about 50 mM to about 800 mM.

27. The method of any one of claim 4 or 7-26, wherein the washing solution comprises a Tris buffer.

28. The method of any one of claim 4 or 7-27, wherein the washing solution comprises EDTA.

29. The method of any one of claim 4 or 7-28, wherein the resuspension solution comprises an RNase inhibitor.

30. The method of any one of claim 4 or 7-29, wherein the washing solution comprises a DNase.

31. The method of claim 30, wherein the DNase is a DNase I.

32. The method of any one of claim 4 or 7-31, wherein the washing solution comprises an RNase III.

33. The method of any one of claim 4 or 7-32, wherein the washing solution comprises a protease.

34. The method of claim 33, wherein the protease is proteinase K.

35. The method of any one of claim 5 or 7-34, wherein the second filter membrane is a tangential flow filtration (TFF) membrane.

36. The method of any one of claim 5 or 7-34, wherein the second filter membrane is a hollow fiber membrane.

37. The method of any one of claim 5 or 7-36, wherein the second filter membrane has a molecular weight cutoff of about 20 kDa to about 150 kDa.

38. The method of any one of claims 7-37, further comprising repeating the steps of (iii)(a) and (iii)(b).

39. The method of claim 38, wherein the steps of (iii)(a) and (iii)(b) are each performed 2, 3, 4, 5, 6, 7, 8, 9, 10, 11, 12, 13, 14, 15, 16, 17, 18, 19, 20, 21, 22, 23, 24, 25, 26, 27, 28, 29, 30, or more, times prior to step (iv).

40. The method of claim 38 or 39, wherein one or more repeats of step (iii)(a) uses a different washing solution than the first iteration of step (iii)(a).

41. The method of any one of claims 1-40, wherein the method comprises, prior to resuspending the mRNA, contacting the precipitated mRNA composition with a salt, surfactant, protease, DNase, and RNase III.

42. The method of any one of claims 7-41, wherein the resuspension solution has a salt concentration of 20 mM or less, 15 mM or less, 10 mM or less, or 5 mM or less.

43. The method of any one of claims 7-42, wherein the resuspension solution comprises Tris.

44. The method of any one of claims 7-43, wherein the resuspension solution comprises EDTA.

45. The method of any one of claims 7-44, wherein the resuspension solution comprises an RNase inhibitor.

46. The method of any one of claims 7-45, wherein the resuspension solution has a pH of about 5.0 to about 7.5.

47. The method of any one of claims 7-46, wherein the resuspension solution has a conductivity of 10 mS/cm or less, 8 mS/cm or less, 6 mS/cm or less, 5 mS/cm or less, 4 mS/cm or less, 3 mS/cm or less, 2.5 mS/cm or less, 2.0 mS/cm or less, 1.5 mS/cm or less, 1.0 mS/cm or less, or 0.5 mS/cm or less.

48. A purified mRNA composition produced by the method of any one of claims 1-47, comprising a resuspended mRNA, wherein the resuspended mRNA comprises an open reading frame encoding a vaccine antigen or therapeutic protein.

49. The purified mRNA composition of claim 48, wherein the purified mRNA composition has a salt concentration of 20 mM or less, 15 mM or less, 10 mM or less, or 5 mM or less.

50. The purified mRNA composition of claim 48 or 49, wherein 1% or fewer, 0.8% or fewer, 0.6% or fewer, 0.5% or fewer, 0.4% or fewer, 0.3% or fewer, 0.2% or fewer, 0.1% or fewer, 0.05% or fewer, 0.04% or fewer, 0.03% or fewer, 0.02% or fewer, or 0.01% or fewer of the mRNA molecules in the purified mRNA composition are double-stranded RNA (dsRNA) molecules.

51. The purified mRNA composition of any one of claims 48-50, wherein the concentration of double-stranded RNA (dsRNA) in the isolated mRNA composition is 1% (w/w) or less, 0.9% or less, 0.8% or less, 0.7% or less, 0.6% or less, 0.5% or less, 0.4% or less, 0.3% or less, 0.2% or less, 0.1% or less, 0.05% or less, 0.04% or less, 0.03% or less, 0.02% or less, 0.01% or less, or 0.008% or less, 0.006% or less, 0.004% or less, 0.002% or less, or 0.001% or less.

52. The purified mRNA composition of any one of claims 48-51, wherein the purified mRNA composition has a protein concentration of 1% (% w/w) or less, 0.8% or less, 0.6% or less, 0.4% or less, or 0.2% or less.

53. The purified mRNA composition of any one of claims 48-52, wherein the purified mRNA composition has a DNA concentration of 1% (% w/w) or less, 0.8% or less, 0.6% or less, 0.4% or less, or 0.2% or less.

54. The purified mRNA composition of any one of claims 48-53, wherein at least 80%, at least 85%, at least 90%, at least 95%, at least 95%, at least 96%, at least 97%, at least 98%, at least 99%, or up to 100% of the mRNAs of the purified mRNA composition comprise a poly(A) tail.
